(19) United States
(12) Patent Application Publication
(10) Pub. No.: US 2021/0403903 A1
(43) Pub. Date: Dec. 30, 2021

(54) MICROBEADS FOR TAGLESS ENCODED CHEMICAL LIBRARY SCREENING

(71) Applicant: NANNA THERAPEUTICS LIMITED, Cambridgeshire (GB)

(72) Inventors: David Hugh WILLIAMS, Cambridgeshire (GB); Stuart Robert WOOD, Cambridgeshire (GB); Nicola THOMPSON, Hertfordshire (GB)

(73) Assignee: NANNA THERAPEUTICS LIMITED, Cambridgeshire (GB)

(21) Appl. No.: 17/288,204

(22) PCT Filed: Oct. 24, 2019
(86) PCT No.: PCT/EP2019/079095

§ 371 Date: Apr. 23, 2021

(30) Foreign Application Priority Data
Oct. 24, 2018 (GB) 1817321.1

(51) Int. Cl. C12N 15/10; B01J 19/00; C12Q 1/6818

(52) U.S. Cl. CPC C12N 15/1065; C12N 15/1068; C12N 15/1075; B01J 19/0046; B01J 2219/00576; B01J 2219/00454; B01J 2219/00466; B01J 2219/005; C12Q 1/6818

(57) ABSTRACT

Disclosed is an encoded chemical library microbead, which microbead has immobilized thereon and/or therein: (i) an encoding tag; and (ii) a target assay system reporter moiety, wherein the reporter moiety exists in a first state in the absence of activity against the target and in a second state in the presence of said activity, and wherein said microbead further comprises a clonal population of one or more chemical structure(s) releasably linked thereto and encoded by said tag.

### FIELD OF THE INVENTION

The present invention relates to microbeads and microcompartments (such as microdroplets) containing them, and to encoded chemical libraries based thereon. The invention also relates to methods for screening the encoded libraries.

### BACKGROUND TO THE INVENTION

Drug discovery typically involves the assembly of large libraries of chemical compounds followed by an assay or screen in which the compounds are added individually to microwells that contain a target to identify “hits” which display a desired activity on the target (e.g. enzymatic activity or displacement of a label). This process is known as high-throughput screening (HTS). Although it can be automated using robotic equipment to test millions of chemicals, it is both laborious and expensive.

There is therefore a fundamental problem stemming from the fact that increased library size increases the screening burden: due to the discrete nature of screening assays, screening time and cost scale approximately linearly with library size. This has imposed severe practical constraints on the size of the chemical libraries screenable using such approaches: HTS is typically applied to libraries containing 10³-10⁶ members.

This problem has been addressed by the development of screening techniques based on selection (e.g. panning techniques). Here, all of the compounds in the library are simultaneously tested for their ability to interact with a target of interest in a one-pot format. In such assays, the time and cost of the screening step is independent of library size, and so the assay can be applied to relatively large libraries. Libraries containing up to 10¹² members have been screened using such approaches.

The problem has also been addressed by the development of microdroplet-based libraries which can be processed by microfluidic techniques to increase throughput by several orders of magnitude (see e.g. Clausell-Tormos et al. (2008) Chemistry & Biology 15: 427-437).

However, both selection-based assays and microdroplet-based screens require that the identity of the selected chemical compounds (i.e. the “hits”) be readily determined: libraries that are screenable but not decodable are not useful.

A solution to this problem was first proposed in 1992 by Brenner and Lerner (Brenner and Lerner (1992) Proc. Natl. Acad. Sci. USA. 89: 5381-5383), and is based on the generation of DNA encoded chemical libraries (DECLs). In a DECL, each compound is linked (tagged) with a DNA sequence which corresponds to its structure or reaction history, thereby serving as a unique identifier of that particular compound (i.e. the DNA tag “encodes” that compound, so serving as a molecular “barcode”).

The compounds can be tagged in various different ways, and it is also possible to use the DNA tag not just to encode a specific chemical structure (“DNA recording”), but also as a template which directs its synthesis (“DNA templating”). The technology has been recently reviewed by Mannocci et al. (2011) Chem. Commun., 47: 12747-12753; Kleiner et al. (2011) Chem Soc Rev. 40(12): 5707-5717; and Mullard (2016) Nature 530: 367-369.

DECL technology is now well-established within the pharmaceutical industry: in 2007, GSK acquired one of the firms that pioneered DECLs, Praecis Pharmaceuticals, for US$55 million, while other top-ten Pharma have started their own in-house DNA-encoded-library programmes. Other biotech companies, including X-Chem, Vipergen, Ensemble Therapeutics and Philochem are also actively developing and exploiting DECL technology.

However, the utility of current DECLs are currently limited by problems associated with the presence of the tag and the nature of the hit assessment, which is solely via binding activity. For example, the encoding tag may: (a) chemically or sterically interfere with the access of the tagged compounds to molecular binding sites on targets of interest, so limiting the number and/or type of hits recovered; (b) limit cellular permeability and/or diffusion, effectively preventing cellular uptake and excluding the use of cell-based phenotypic screens which rely on access to the cytoplasm; (c) limit the extent to which the chemical compounds can be chemically modified after tagging (certain reactions being chemically incompatible with the tag); and (d) limit the usefulness of structure-activity analyses, since such analyses are confounded by the potential impact of the tag itself on activity and the fact that only target-binding, rather than functional (e.g. enzyme) activity, is detected.

There is therefore a need for HTS techniques which permit the screening of decodable chemical libraries and which address the foregoing problems.

### SUMMARY OF THE INVENTION

According to a first aspect of the present invention, there is provided an encoded chemical library microbead, which microbead has immobilized thereon and/or therein: (i) an encoding tag; and (ii) a target assay system reporter moiety, wherein the reporter moiety exists in a first state in the absence of activity against the target and in a second state in the presence of said activity, and wherein said microbead further comprises a clonal population of one or more chemical structure(s) releasably linked thereto and encoded by said tag.

According to a second aspect of the present invention, there is provided a chemical library microcompartment which contains a microbead of the invention and an aqueous solvent.

According to a third aspect of the present invention, there is provided an encoded chemical library (ECL) comprising a plurality of microcompartments of the invention, wherein each of the microcompartments contains a different chemical structure.

According to a fourth aspect of the present invention, there is provided a method for screening an ECL of the invention for chemical structures having activity against a target, the method comprising the steps of:
(a) providing said ECL;
(b) releasing the chemical structures from the microbeads to produce a plurality of free, tagless chemical structures (TCSs) dissolved in the solvent and contained within microcompartments together with the microbeads from which they were released, such that a spatial association between each TCS and its encoding tag is maintained;
(c) assaying the TCSs by incubating the ECL microcompartments of step (b) under conditions such that the state of the reporter moieties immobilized in or on the microbeads contained therein is determined by the level of activity against the target;
(d) releasing the microbeads by opening the microcompartments; and
(e) screening the released microbeads by determining the state of the reporter moieties, whereby chemical structures having activity against the target can be identified by decoding the tags of microbeads having reporter moieties in the second state.

Other aspects, preferred features and preferred operative combinations of features of the invention are defined and described in the numbered paragraphs set out below.

1. An encoded chemical library microbead, which microbead has immobilized thereon and/or therein: (i) an encoding tag; and (ii) a target assay system reporter moiety, wherein the reporter moiety exists in a first state in the absence of activity against the target and in a second state in the presence of said activity, and wherein said microbead further comprises a clonal population of one or more chemical structure(s) releasably linked thereto and encoded by said tag.

2. The microbead of paragraph 1, wherein said encoding tag also encodes the target assay system reporter moiety.

3. The microbead of paragraph 1 or paragraph 2, wherein said encoding tag also encodes the target.

4. The microbead of any one of the preceding paragraphs, wherein the chemical structures are present at a loading of between 1 and 10¹³ molecules per microbead.

5. The microbead of any one of the preceding paragraphs, wherein said microbead comprises a clonal population of a plurality of chemical structures, optionally wherein said encoding tag also encodes the loading of the chemical structures.

6. The microbead of any one of the preceding paragraphs, wherein the microbead has immobilized thereon or therein a plurality of target assay system reporter moieties, optionally wherein said encoding tag also encodes the loading of the reporter moieties.

7. The microbead of paragraph 6, wherein the ratio of reporter moieties in the first state to the second state is correlated with the level of activity against the target.

8. The microbead of any one of the preceding paragraphs, wherein the microbead is substantially spherical.

9. The microbead of paragraph 8, wherein the microbead has a diameter of up to 400 μm.

10. The microbead of paragraph 8, wherein the microbead has a diameter of 1-100 μm.

11. The microbead of paragraph 8, wherein the microbead has a diameter of <50 μm.

12. The microbead of any one of the preceding paragraphs, wherein the microbead is formed of a hydrogel or polymer.

13. The microbead of paragraph 12, wherein the microbead is formed of a solid, for example silicone, a polymer for example polystyrene, polypropylene and divinylbenzene or hydrogels, for example selected from agarose, alginate, polyacrylamide and polylactic acid.

14. The microbead of any one of the preceding paragraphs, wherein the encoding tag comprises a nucleic acid.

15. The microbead of paragraph 14, wherein the nucleic acid is DNA.

16. The microbead of any one of paragraphs 1-14, wherein the microbead comprises non-DNA tags, non-RNA tags, modified nucleic acid tags, peptide tags, light-based barcodes (e.g. quantum dots), RFID tags, reporter chemicals linked by click chemistry and mass spectrometry-decodable tags.

17. The microbead of any one of the preceding paragraphs, wherein the chemical structures are small molecules.

18. The microbead of any one of the preceding paragraphs, wherein the chemical structures are releasably linked to the microbead by a cleavable linker.

19. The microbead of paragraph 18, wherein the linker is scarless, such that the chemical structure(s) can be cleaved from the microbead in a form in which they are completely or substantially free of linker residues.

20. The microbead of paragraph 18 or 19, wherein the cleavable linker comprises a linker selected from: enzymatically cleavable linkers; chemically cleavable linkers; photocleavable linkers and combinations of two or more of the foregoing.

21. The microbead of any one of paragraphs 18-20 wherein the cleavable linker is selected from: nucleophile/base-sensitive linkers; reduction sensitive linkers; UV-sensitive linkers; electrophile/acid-sensitive linkers; metal-assisted cleavage-sensitive linkers; oxidation-sensitive linkers; and combinations of two or more of the foregoing.

22. The microbead of any one of paragraphs 18-20 wherein the cleavable linker is an enzymatically cleavable linker, for example a being cleavable by an enzyme selected from proteases (including enterokinases), nucleases, nitroreductases, phosphatases, β-glucuronidase, lysosomal enzymes, TEV, trypsin, thrombin, cathepsin B, B and K, caspase, matrix metalloproteinases, phosphodiesterases, phospholipidases, esterases, reductases and β-galactosidases.

23. The microbead of any one of paragraphs 18-20 wherein the cleavable linker comprises RNA and wherein the chemical structures can be released by contact with an RNase.

24. The microbead of any one of paragraphs 18-20 wherein the cleavable linker comprises a peptide and wherein the chemical structures can be released by contact with a peptidase.

25. The microbead of any one of paragraphs 18-20 wherein the cleavable linker comprises DNA and wherein the chemical structures can be released by contact with a site-specific endonuclease.

26. The microbead of any one of paragraphs 18-20 wherein the cleavable linker is a self-immolative linker comprising a cleavage moiety and a self-immolative moiety (SIM), optionally wherein the cleavage moiety is a peptide or non-peptide enzymatically cleavable moiety, e.g. Val-Cit-PAB.

27. The microbead of any one of the preceding paragraphs, wherein the chemical structures are indirectly or directly linked to the microbead.

28. The microbead of paragraph 27, wherein the chemical structures are indirectly linked to the microbead via the encoding tag.

29. The microbead of paragraph 28, wherein the chemical structures are linked to the encoding tag by nucleic acid hybridization.

30. The microbead of any one of the preceding paragraphs, wherein the target is an enzyme, optionally a mammalian (e.g. human), bacterial or viral enzyme, for example an enzyme selected from: proteases; kinases; dehydrogenases and phosphatases.

31. The microbead of paragraph 30 wherein the target assay system reporter moiety is: (a) a substrate, inhibitor, activator or chaperone of the enzyme; or (b) the enzyme or a fragment thereof.

32. The microbead of paragraph 31 wherein the target assay system reporter moiety comprises: (a) a catalytic site of the enzyme; and/or (b) an allosteric site of the enzyme.

33. The microbead of any one of paragraphs 1-29, wherein the target is a protein, for example a mammalian (e.g. human), bacterial or viral protein.

34. The microbead of paragraph 33 wherein the target assay system reporter moiety is: (a) a binding partner of the protein; or (b) the protein or a fragment thereof.

35. The microbead of any one of paragraphs 1-29, wherein the target is a receptor, for example a mammalian (e.g. human), bacterial or viral protein.

36. The microbead of paragraph 35 wherein the target assay system reporter moiety is: (a) a ligand of the receptor; or (b) the receptor or a fragment thereof.

37. The microbead of any one of paragraphs 1-29, wherein the target is a receptor ligand.

38. The microbead of paragraph 37 wherein the target assay system reporter moiety is: (a) the receptor ligand or a fragment thereof; or (b) the receptor or a fragment thereof.

39. The microbead of any one of paragraphs 1-29, wherein the target is an enzyme substrate.

40. The microbead of paragraph 39 wherein the target assay system reporter moiety is: (a) the enzyme substrate; or (b) the enzyme, or a fragment thereof.

41. The microbead of any one of paragraphs 1-29, wherein the target is a molecular chaperone.

42. The microbead of paragraph 41 wherein the target assay system reporter moiety is: (a) the chaperone or a fragment thereof; or (b) the chaperoned molecule, for example a chaperone-binding peptide.

43. The microbead of any one of paragraphs 1-29, wherein the target is a toxin.

44. The microbead of paragraph 43 wherein the target assay system reporter moiety is: (a) the toxin or a fragment thereof; or (b) a binding partner of the toxin.

45. The microbead of any one of paragraphs 1-29, wherein the target is a drug.

46. The microbead of paragraph 45 wherein the target assay system reporter moiety is: (a) the drug or a fragment thereof; or (b) a binding partner of the drug.

47. The microbead of any one of paragraphs 1-29, wherein the target assay system reporter moiety is turned over and chemical structures having catalytic activity can be identified by decoding the tags of microbeads having reporter moieties in the turned over state.

48. The microbead of any one of paragraphs 1-29, wherein the target assay system reporter moiety is fluorescent and chemical structures having quenching activity can be identified by decoding the tags of microbeads having reporter moieties in the quenched state.

49. The microbead of any one of paragraphs 1-29, wherein the target assay system reporter moiety is non-fluorescent substrate and chemical structures which function as fluorophore coatings can be identified by decoding the tags of microbeads having fluorescent reporter moieties.

50. The microbead of any one of paragraphs 1-29, wherein the target assay system reporter moiety is substrate and chemical structures which function as chromophore coatings can be identified by decoding the tags of microbeads having chromatic reporter moieties.

51. The microbead of any one of paragraphs 1-29, wherein the target assay system reporter moiety is substrate and chemical structures which function as a substrate coating can be identified by decoding the tags of microbeads having coated reporter moieties.

52. The microbead of any one of the preceding paragraphs, wherein the first and second states of the target assay system reporter moiety are distinguished by: (a) fluorescence, for example quenched or unquenched fluorescence; and/or (b) cleaved or uncleaved conformation; and/or (c) phosphorylated or non-phosphorylated states; (d) different glycosylation type, pattern or extent; and/or (e) different antigenic determinants; and/or (f) being bound or unbound to a ligand; and/or (g) being complexed or uncomplexed with one or more further assay system components.

53. A chemical library microcompartment which contains a microbead as defined in any one of the preceding paragraphs and a solvent, for example an aqueous solvent.

54. The microcompartment of paragraph 53 which further contains a cleaving agent for releasing the chemical structure(s) from the microbead into solution, optionally wherein the cleaving agent is an enzyme, for example an enzyme selected from proteases (including enterokinases), nucleases, nitroreductases, phosphatases, β-glucuronidase, lysosomal enzymes, TEV, trypsin, thrombin, cathepsin B, B and K, caspase, matrix metalloproteinases, phosphodiesterases, phospholipidases, esterases and β-galactosidases.

55. The microcompartment of any one of paragraphs 53-54 which is in the form of a microdroplet, microparticle or microvesicle, optionally being a microdroplet of a water-in-oil emulsion having a surfactant-stabilized interface.

56. The microcompartment of any one of paragraphs 53-55 wherein the chemical structures are present at a concentration of at least: 0.1 nM, 0.5 nM, 1.0 nM 5.0 nM, 10.0 nM, 15.0 nM, 20.0 nM, 30.0 nM, 50.0 nM, 75.0 nM, 0.1 μM, 0.5 μM, 1.0 μM, 5.0 μM, 10.0 μM, 15.0 μM, 20.0 μM, 30.0 μM, 50.0 μM, 75.0 μM, 100.0 μM, 200.0 μM, 300.0 μM, 500.0 μM, 1 mM, 2 mM, 5 mM or 10 mM.

57. The microcompartment of any one of paragraphs 53-56 which is substantially spherical.

58. The microcompartment of paragraph 57 which has a diameter of from 1 to 500 μm, optionally less than <100 μm.

59. The microcompartment of any one of paragraphs 53-58 in which the chemical structure(s) have been released from the microbead to produce free, tagless chemical structure(s) (TCSs) dissolved in the solvent and spatially associated with the encoding tag.

60. The microcompartment of any one of paragraphs 53-59 which further contains one or more additional component(s) of said target assay system.

61. The microcompartment of paragraph 60 wherein the additional component comprises an antibody.

62. The microcompartment of paragraph 61 wherein the antibody specifically binds either the first or second states of the reporter moiety.

63. The microcompartment of paragraph 61 or 62 wherein the antibody is attached to a magnetic bead or detectable label, optionally a fluorescent label.

64. The microcompartment of any one of paragraphs 60-63 wherein the additional component comprises a target selected from those defined in any one of paragraphs 30, 34, 37, 40, 43, 46 and 49, optionally in labelled form.

65. The microcompartment of paragraph 64 wherein:
(a) the additional component comprises a target selected from those defined in any one of paragraphs 30, 34, 37, 40, 43, 46 and 49, optionally in labelled form; and
(b) the target assay system reporter moiety is selected from those defined in any one of paragraphs: 31-33; 35-36; 38-49; 41-42; 44-45; 47-48 and 50-51, respectively.

66. The microcompartment of any one of paragraphs 53-65 which further contains an additional moiety which is dissolved in the solvent and encoded by a second tag immobilized in or on the microbead.

67. The microcompartment of paragraph 66 which is obtainable, or obtained by, co-encapsulation of a microbead as defined in any one of paragraphs 1-52 with said additional moiety and second encoding tag, followed by immobilization of the second tag on or in the microbead.

68. The microcompartment of paragraph 67 wherein the encoding tags are DNA tags and the second encoding tag is ligated to the tag encoding the chemical structure after co-encapsulation.

69. An encoded chemical library (ECL) comprising a plurality of microcompartments as defined in any one of paragraphs 53-68, wherein each of the microcompartments contains a different chemical structure.

70. The ECL of paragraph 69 which comprises a number n of different clonal populations of chemical structures, each clonal population being confined to n discrete library microcompartments.

71. The ECL of paragraph 70 wherein: (a) n>10³; or (b) n>10⁴; or (c) n>10⁵; or (d) n>10⁶; or (e) n>10⁷; or (f) n>10⁸; or (g) n>10⁹; or (h) n>10¹⁰; or (i) n>10¹¹; (j) n>10¹²; (k) n>10¹³; (l) n>10¹⁴; or (m) n>10¹⁵.

72. The ECL of paragraph 71 wherein n=10⁶ to 10⁹.

73. A method for screening an ECL as defined in any one of paragraphs 69-72 for chemical structures having activity against a target, the method comprising the steps of:
(a) providing said ECL;
(b) releasing the chemical structures from the microbeads to produce a plurality of free, tagless chemical structures (TCSs) dissolved in the solvent and contained within microcompartments together with the microbeads from which they were released, such that a spatial association between each TCS and its encoding tag is maintained;
(c) assaying the TCSs by incubating the ECL microcompartments of step (b) under conditions such that the state of the reporter moieties immobilized in or on the microbeads contained therein is determined by the level of activity against the target;
(d) releasing the assayed microbeads by opening the microcompartments; and
(e) screening the released and assayed microbeads by determining the state of the reporter moieties, whereby chemical structures having activity against the target can be identified by decoding the tags of microbeads having reporter moieties in the second state.

74. The method of paragraph 73 wherein step (a) comprises the step of nucleic acid-recorded, for example DNA-recorded, synthesis of the chemical structures.

75. The method of paragraph 73 wherein step (a) comprises the step of split-and-pool nucleic acid-recorded synthesis of the chemical structures.

76. The method of any one of paragraphs 73-75 wherein step (c) comprises incubation in an homogeneous aqueous phase assay system.

77. The method of any one of paragraphs 73-76 wherein step (d) further comprises stopping the incubation, for example by heat denaturation, freezing, addition of inhibitors or breaking of the microcompartments.

78. The method of paragraph 77 wherein the microcompartments are broken by centrifugation, sonication and/or filtration or by the addition of solvents and/or surfactants.

79. The method of any one of paragraphs 73-78 further comprising the step of isolating the microbeads released in step (d) during or prior to screening step (e).

80. The method of any one of paragraphs 73-79 wherein the screening step comprises fractionation and/or selection of the released and assayed microbeads.

81. The method of paragraph 80 wherein the screening step comprises FRET, FACS, immunoprecipitation, immunosedimentation, immunofiltration, affinity column chromatography and/or magnetic microbead affinity selection high throughput screening.

82. The method of any one of paragraphs 73-81 wherein the screening step (e) comprises determining the level of activity against the target by measuring the ratio of reporter moieties in the first state to the second state.

83. The method of any one of paragraphs 73-82 wherein the microbead comprises a clonal population of a plurality of chemical structures and said encoding tag also encodes the loading of the chemical structures, and wherein the screening step (e) comprises determining the level of activity against the target by correlating the loading of the chemical structures with the ratio of reporter moieties in the first state to the second state.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the terms “consist essentially of” is used herein to define specified integer(s), features or steps as well as those which do not materially affect the character or function of the defined integer(s), features or steps (while excluding other integers, features or steps which do materially affect the character or function of the defined integer(s), features or steps). As used herein, the term “consisting” is used to indicate the presence of the recited integer(s), features or steps (e.g. an element, characteristic, property, method/process step or limitation) or group of integers (e.g. features, element, characteristics, properties, method/process steps or limitations) alone and to exclude other integer(s), features or steps.

As used herein, the term assay system defines means for detecting activity against a target. The target is typically a drug target, and so may be a molecule (such as a protein) relevant to a disease. The assay system directly or indirectly produces a detectable and/or measurable signal when one or more components thereof contact or react with a chemical structure present in the library and having the desired activity. Any assay system may be used according to the invention, provided that it comprises an immobilized target assay system reporter moiety, wherein the reporter moiety exists in a first state in the absence of activity against the target and in a second state in the presence of said activity. In some cases, the assay system may consist, or consist essentially of, the immobilized target assay system reporter moiety (i.e. the assay system does not include components other than the immobilized reporter moiety), for example in embodiments where the free chemical structure interacts directly with the reporter moiety (e.g. by selectively binding to it) or where the reporter moiety alone and itself signals an interaction with the free chemical structure (e.g. by autophosphorylating, switching conformation, fluorescing or quenching fluorescence in the presence of the free chemical structure).

The desired activity may be target protein binding, pharmacological activity, cell receptor binding, antibiotic, anticancer, antiviral, antifungal, antiparasitic, pesticide, pharmacological, immunological activity, production of any desired compound, increased production of a compound or breakdown of a specific product. The desired activity may be activity against a pharmacological target cell, cell protein or metabolic pathway. The desired activity may also be the ability to modulate gene expression, for example by decreasing or increasing the expression of one or more gene(s) and/or their temporal or spatial (e.g. tissue-specific) expression patterns. The desired activity may be binding activity, for example to act as a ligand to a target protein. The desired activity may also be one which is useful in various industrial processes, including bioremediation, microbially-enhanced oil recovery, sewage treatment, food production, biofuel production, energy generation, bio-production, bio-digestion/biodegradation, vaccine production and probiotic production. It could also be a chemical agent, such as a fluorophore, or pigment, specific chemical reaction or any chemical reaction that can be tied to a colour, matrix structure or refractive index change.

The assay system may comprise chemical indicators, including reporter molecules and detectable labels (as herein defined). It may be, for example, colorimetric (i.e. result in a coloured reaction product that absorbs light in the visible range), fluorescent (e.g. based on an enzyme converts a substrate to a reaction product that fluoresces when excited by light of a particular wavelength) and/or luminescent (e.g. based on bioluminescence, chemiluminescence and/or photoluminescence).

The assay system may comprise cells, for example the target cells as described herein. The assay system may also comprise proteins, for example the target proteins as described herein. Alternatively, or in addition, the assay system may comprise cell fractions, cellular components, tissue, tissue extracts, multi-protein complexes, membrane-bound proteins membrane fractions and/or organoids.

The term ligand as used herein to define a binding partner for a biological target molecule in vivo (for example, an enzyme or receptor). Such ligands therefore include those which bind (or directly physically interact) with the target in vivo irrespective of the physiological consequences of that binding. Thus, the ligands of the invention may bind the target as part of a cellular signalling cascade in which the target forms a part. Alternatively, they may bind the target in the context of some other aspect of cellular physiology. In the latter case, the ligands may for example bind the target at the cell surface without triggering a signalling cascade, in which case the binding may affect other aspects of cell function. Thus, the ligands of the invention may bind the target at the cell surface and/or intracellularly.

As used herein, the term small molecule means any molecule having a molecular weight of 1500 Da or less, preferably 1000 Da or less, for example less than 900 Da, less than 800 Da, less than 600 Da or less than 500 Da. Preferably, the chemical structures present in the libraries of the invention may be small molecules as herein defined, particularly small molecules having a molecular weight of less than 600 Da.

As used herein, the term large molecule means any molecule having a molecular weight greater than 1500 Da. Such molecules may include, for example, macrocycles and peptides (which typically have molecular weights in the kDa range) as well as antibodies and proteins (which may have molecular weights in the 100's of kDa range).

As used herein, the term antibody defines whole antibodies (including polyclonal antibodies and monoclonal antibodies (mAbs)). The term is also used herein to refer to antibody fragments, including F(ab), F(ab′), F(ab′)2, Fv, Fc3 and single chain antibodies (and combinations thereof), which may be produced by recombinant DNA techniques or by enzymatic or chemical cleavage of intact antibodies. The term “antibody” is also used herein to cover bispecific or bifunctional antibodies which are synthetic hybrid antibodies having two different heavy/light chain pairs and two different binding sites. Bispecific antibodies can be produced by a variety of methods including fusion of hybridomas or linking of Fab′ fragments. Also covered by the term “antibody” are chimaeric antibodies (antibodies having a human constant antibody immunoglobulin domain coupled to one or more non-human variable antibody immunoglobulin domain, or fragments thereof). Such chimaeric antibodies therefore include “humanized” antibodies. Also covered by the term “antibody” are minibodies (see WO 94/09817), single chain Fv-Fc fusions and human antibodies produced by transgenic animals. The term “antibody” also includes multimeric antibodies and higher-order complexes of proteins (e.g. heterodimeric antibodies).

As used herein, the terms peptide, polypeptide and protein are used interchangeably to define organic compounds comprising two or more amino acids covalently joined by peptide bonds. The corresponding adjectival term “peptidic” is to be interpreted accordingly. Peptides may be referred to with respect to the number of constituent amino acids, i.e., a dipeptide contains two amino acid residues, a tripeptide contains three, etc. Peptides containing ten or fewer amino acids may be referred to as oligopeptides, while those with more than ten amino acid residues are polypeptides. Such peptides may also include any of the modifications and additional amino and carboxy groups.

As used herein, the term click chemistry is a term of art introduced by Sharpless in 2001 to describe reactions that are high yielding, wide in scope, create only by-products that can be removed without chromatography, are stereospecific, simple to perform and can be conducted in easily removable or benign solvents. It has since been implemented in many different forms, with wide applications in both chemistry and biology. A subclass of click reactions involve reactants which are inert to the surrounding biological milieu. Such click reactions are termed bioorthogonal. Bioorthogonal reactant pairs suitable for bioorthogonal click chemistry are molecular groups with the following properties: (1) they are mutually reactive but do not significantly cross-react or interact with cellular biochemical systems in the intracellular milieu; (2) they and their products and by-products are stable and nontoxic in physiological settings; and (3) their reaction is highly specific and fast. The reactive moieties (or click reactants) may be selected by reference to the particular click chemistry employed, and so any of a wide range of compatible pairs of bioorthogonal click reactants known to those skilled in the art may be used according to the invention, including Inverse electron demand Diels-Alder cycloaddition reaction (IEDDA), Strain-promoted alkyne azide cycloaddition (SPAAC) and Staudinger ligation.

The term isolated (and related terms) is used herein in relation to any material (e.g. a chemical compound, assay reagent, microbead, target protein or target cell) to indicate that the material exists in a physical milieu distinct from that in which it occurs in nature (or in which it occurred prior to isolation). For example, isolation of microbeads released from microcompartments may comprise simply separating the beads from one or more of the physicochemical components of the opened (e.g. broken) microcompartments, for example by separation from one or more of: (a) free, tagless chemical structure(s); and/or (b) the solvent; and/or (c) cleaving agent(s); and/or (d) additional components of the target assay system For example, isolated cells may be substantially isolated (for example purified) with respect to the complex tissue milieu in which they naturally occur. Isolated cells may, for example, be purified or separated. In such cases, the isolated cells may constitute at least 60%, 70%, 75%, 80%, 85%, 90%, 95%, 96%, 97%, 98%, 99%, or 100% of the total cell types present. Isolated cells may be obtained by routine techniques known to those skilled in the art, including FACS, density gradient centrifugation, enrichment culture, selective culture, cell sorting and panning techniques using immobilized antibodies against surface proteins.

When the isolated material is purified, the absolute level of purity is not critical and those skilled in the art can readily determine appropriate levels of purity according to the use to which the material is to be put. Preferred, however, are purity levels of at least 60%, 70%, 75%, 80%, 85%, 90%, 95%, 96%, 97%, 98%, 99%, or 100% w/w. In some circumstances, the isolated material forms part of a composition (for example a more or less crude cellular extract containing many other cellular components) or buffer system, which may contain other components.

The term contacting and related terms are used herein refers to the process of allowing at least two distinct moieties or systems (e.g. chemical structures and a component of an assay system, such as the reporter moiety) to become sufficiently proximal to react, interact, physically touch or bind.

The term detectable label is used herein to define a moiety detectable by spectroscopic, fluorescent, photochemical, biochemical, immunochemical, chemical, electrochemical, radiofrequency or by any other physical means. Suitable labels include fluorescent proteins, fluorescent dyes, electron-dense reagents, enzymes (e.g., as commonly used in an ELISA), biotin, digoxigenin, or haptens and proteins or other entities which can be made detectable, for example by incorporating a radio- or fluorescent label into a peptide or antibody specifically reactive with a target peptide.

As used herein, the term microbead is used to define a solid (e.g. polymer, polystyrene) or hydrogel (e.g. alginate) particle having a longest dimension of up to 400 μm, preferably 1-100 μm and more preferably less than 50 μm. Preferred are substantially spherical particles having a diameter of up to 400 μm, preferably 1-100 μm and more preferably less than 50 μm. Preferred microbeads are formed from gels (including hydrogels such as agarose), for example by fragmentation of a gelled bulk composition or moulding from a pre-gelled state.

As used herein, the term microcompartment as applied to the chemical library of the invention defines any structure which can contain or encapsulate the microbeads of the invention and maintain a spatial association between a free chemical structure and the microbead from which it was released. The microcompartments of the invention therefore serve as closed reaction chambers containing a solvent in which free, tagless chemical structures are spatially associated with their cognate encoding tags, along with the target assay system reporter moiety, any additional component(s) of the target assay system and/or cleaving agents. Microcompartments suitable for use according to the invention are conveniently achieved by micro-compartmentalization, which is a process of physically confining the microbeads. Physical confinement can be achieved through the use of various micro-compartments, including microdroplets, microparticles and microvesicles, as explained below.

As used herein, the term microdroplet defines a small, discrete volume of a fluid, liquid or gel having a diameter of 0.1 μm to 1000 μm and/or a volume of between 5×10⁻⁷ pL and 500 nL. Typically, the microdroplets have a diameter of less than 1000 μm, for example less than 500 μm, less than 500 μm, less than 400 μm, less than 300 μm, less than 200 μm, less than 100 μm, less than 50 μm, less than 40 μm, less than 30 μm, less than 20 μm, less than 10 μm, less than 5 μm, or less than 1 μm. Thus, the microdroplets may be substantially spherical with a diameter of: (a) less than 1 μm; (b) less than 10 μm; (c) 0.1-10 μm; (d) 10 μm to 500 μm; (b) 10 μm to 200 μm; (c) 10 μm to 150 μm; (d) 10 μm to 100 μm; (e) 10 μm to 50 μm; or (f) about 100 μm.

The microdroplets of the invention are typically comprised of an isolated portion of a first fluid, liquid or gel that is completely surrounded by a second fluid, liquid or gel (e.g. an immiscible liquid or a gas). In some cases, the droplets may be spherical or substantially spherical. However, in some cases, the microdroplets may be non-spherical and have an irregular shape (for example due to forces imposed by the external environment or during physical manipulation during the assay and screening processes described herein). Thus, microdroplets may be substantially cylindrical, plug-like and or oval in shape (for example in circumstances where they conform to the geometry of a surrounding microchannel).

As used herein, the term microparticle defines a particle having a diameter of less than 1000 μm, for example less than 500 μm, less than 500 μm, less than 400 μm, less than 300 μm, less than 200 μm, less than 100 μm, less than 50 μm, less than 40 μm, less than 30 μm, less than 20 μm, less than 10 μm, less than 5 μm, or less than 1 μm. The microparticles are preferably non-planar and may have a largest dimension of (or be substantially spherical with a diameter of): (a) 10 μm to 500 μm; (b) 10 μm to 200 μm; (c) 10 μm to 150 μm; (d) 10 μm to 100 μm; (e) 10 μm to 50 μm; or (f) about 100 μm. Microparticles may therefore be encapsulated within microdroplets as herein defined.

Microparticles may be formed from rigid solids, flexible gels, porous solids, porous gels or networks or matrices of rigid or semi-rigid fibrils or tubules.

The term microvesicle is used herein to define a hollow microparticle comprising an outer wall or membrane enclosing an internal volume, such as a liposome.

The microdroplets, microparticles and microvesicles of the invention may be monodisperse. The term monodisperse, as applied to the microdroplets and microparticles for use according to the invention, defines a microdroplet/microparticle population having a coefficient of droplet/particle size dispersion, c, of not more than 1.0, not more than 0.5, and preferably not more than 0.3. Said coefficient c is defined by the following equation:

ε=(⁹⁰Dₚ−¹⁰Dₚ)/⁵⁰Dₚ (1)

where ¹⁰Dₚ, ⁵⁰Dₚ and ⁹⁰Dₚ are the particle sizes when the cumulative frequencies estimated from a relative cumulative particle size distribution curve for the emulsion are 10%, 50% and 90%, respectively. The case where ε=0 means an ideal state in which emulsion particles show no particle size scattering at all.

As used herein, the term encoding tag is used, in relation to a chemical structure, to define a moiety or agent which contains information which uniquely identifies the chemical structure or its reaction history, thereby serving as a unique identifier of that particular chemical structure (i.e. the tag “encodes” that structure and serves as a molecular “barcode”). The information may be encoded in any form, but in preferred embodiments the tag is a nucleic acid (for example DNA) tag in which the information is encoded in the sequence of the nucleic acid. However, other tags may be used, including non-DNA tags, non-RNA tags, modified nucleic acid tags, peptide tags, light-based barcodes (e.g. quantum dots) and RFID tags.

As used herein, the term clonal in relation to a population of chemical structures defines a population of one or more chemical structures each of which is encoded by a common tag. Clonal chemical structures are chemically identical, or may differ only in respect to the nature and/or position of the cleavable linker reversibly linking the chemical structure to the microbead (or in respect to a scar remaining after cleavage of such a linker).

As used herein, the term free as applied to chemical structures is used to define a chemical structure which is not bound to a solid phase or is in solution. In some embodiments, the term defines a chemical structure which is not covalently bound to a solid phase. Free chemical structures may therefore enter a liquid or gel phase and/or a solution, and may in some embodiments interact with one or more components of a target assay system (for example, the immobilized target assay system reporter moiety of the microbead of the invention).

As used herein, the term self-immolative linker defines a linker comprising a self-immolative chemical group (which may be referred to herein as a self-immolative moiety or “SIM”) capable of directly or indirectly (e.g. via a peptide moiety) covalently linking the chemical structure and its encoding tag to form a stable tagged chemical structure, and which is capable of releasing the encoding tag from the chemical structure by a mechanism involving spontaneous release of the chemical structure (for example via an electronic cascade triggered by enzymatic cleavage that leads to the expulsion of a leaving group and release of the free chemical structure).

Microbeads

The microbeads of the invention may have any geometry, and may be spheroidal, spherical, cuboidal, pyramidal, oblong, cylindrical or toroidal. They may be formed of a solid or gel.

Suitable gels include polymer gels, for example polysaccharide or polypeptide gels which can be solidified from a liquid into a gel, for example by heating, cooling or pH adjustment. Other suitable gels include hydrogels, including alginate, gelatine, agarose and self-assembling peptide gels.

Other suitable materials include lipids, peptides, plastics (such as polystyrene, poly(vinyl) chloride, cyclo-olefin copolymers, polyacrylamide, polylactic acid, polyacrylate, polyethylene, polypropylene, poly(4-methylbutene), polymethacrylate, poly(ethylene terephthalate), polytetrafluoroethylene (PTFE), nylon and polyvinyl butyrate.

Thus, the microbeads may be formed of a polymer or a combination of polymers. In such embodiments, the microbeads may comprise a polyester, for example a polyester coupled to a hydrophilic polymer. Here, the polyester may comprise a poly(lactic acid), poly(glycolic acid), poly(lactic-co-glycolic acid), or polycaprolactone and/or the hydrophilic polymer may comprise a polyether (for example comprising polyethylene glycol).

Suitable microparticle materials also include inorganic materials such as silicon, glass, metals and ceramics.

The microbeads may be functionalized with reactive groups or moieties, such as streptavidin, amine, cyanogen bromide or carboxylic acid. Alternatively, the microbeads for use according to the invention can be solid supports, such as those made of silicon, polystyrene (PS), crosslinked poly(styrene/divinylbenzene) (P[S/DVB]) and poly(methyl methacrylate) (PMMA).

Encoding Tags

Any encoding tag may be used according to the invention provided that it contains information (for example, in the form of chemical and/or optical properties/characteristics) which uniquely identifies its cognate chemical structure (or its reaction history), thereby serving as a unique identifier of that particular chemical structure. Thus, the tag “encodes” a particular chemical structure and serves as a molecular “barcode”.

In preferred embodiments the tag is a nucleic acid (for example DNA) tag in which the information is encoded in the sequence of the nucleic acid. However, other tags may be used, including non-DNA tags, non-RNA tags, modified nucleic acid tags, peptide tags, light-based barcodes (e.g. quantum dots) and RFID tags.

As explained above, the chemical structures can be tagged in various different ways, and it is also possible to use the DNA tag not just to encode a specific chemical structure (“DNA recording”), but also as a template which directs its synthesis (“DNA templating”—see below). The technology has been recently reviewed by Mannocci et al. (2011) Chem. Commun., 47: 12747-12753; Kleiner et al. (2011) Chem Soc Rev. 40(12): 5707-5717; and Mullard (2016) Nature 530: 367-369.

The encoding tags may also encode other useful information. For example, the tag may also include information specifying: (a) the loading of the chemical compound on the microbead; and/or (b) the nature and/or loading of the reporter moieties; and/or (c) the nature and/or loading of the target. In such embodiments, the encoding tags may be immobilized on the microbead as separate tags, or present as part of a unitary concatenated tags. In such embodiments, the encoding tags may be functionalized with a plurality of different cross-linking groups, so that tagging can occur at a plurality of distinct crosslinking sites on the microbead.

The use of such tags permits the screening of multiple targets and permits dose response to be established.

The tag may also include information specifying the nature and/or loading of additional moieties microcompartmentalized along with the microbeads. In such embodiments, a large number of additional factors can be incorporated during encapsulation and rapidly screened as part of the library screen. This could be a variety of targets, additional co-factors, compounds or proteins. This can be done by utilising different fluidic channels, pico-injection or droplet merging techniques which are well known to those practiced in the arts.

Tag Sequencing

Any suitable sequencing technique can be used, including Sanger sequencing, but preferred are sequencing methods and platforms termed next-generation sequencing (NGS), also known as high-throughput sequencing. There are many commercially available NGS sequencing platforms that are suitable for use in the methods of the invention. Sequencing-by-synthesis (SBS)-based sequencing platforms are particularly suitable. The Illumina™ system (which generates millions of relatively short sequence reads (54, 75, 100, 150 or 300 bp) is particularly preferred. Here, DNA molecules are first attached to primers on a slide and amplified so that local clonal colonies are formed (bridge amplification). Four types of ddNTPs are added, and non-incorporated nucleotides are washed away. Unlike pyrosequencing, the DNA can only be extended one nucleotide at a time. A camera takes images of the fluorescently labeled nucleotides then the dye along with the terminal 3′ blocker is chemically removed from the DNA, allowing a next cycle.

Other systems capable of short sequence reads include SOLiD™ and Ion Torrent technologies (both sold by Thermo Fisher Scientific Corporation). SOLiD™ technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion Torrent Systems Inc. has developed a system based on using standard sequencing chemistry, but with a novel, semiconductor-based detection system. This method of sequencing is based on the detection of hydrogen ions that are released during the polymerisation of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

Methods such as or similar to that that used by Oxford Nanopore Technologies, where nucleic acid or other macromolecules are passed through a nano scale pore and the specific ionic current changes or electrical signals generated are used to identify it. For example, the individual bases of an oligonucleotide can be identified either as successive bases pass through the ion pore as part of the oligonucleotide or as single nucleotides after successive cleavage steps.

Target Assay System Reporter Moiety

The microbeads of the invention comprise an immobilized target assay system reporter moiety. The reporter moiety functions as such in the context of a chemical library microcompartment which contains the microbead and an aqueous solvent, in which the chemical structure(s) have been released to produce free, tagless chemical structure(s) (TCSs) dissolved in the solvent. In this context, the TCSs are free to contact the target assay system reporter moiety and/or one or more additional component(s) of the target assay system, and the microbead can be assayed in the screening methods of the invention.

Any suitable reporter moiety may be used, provided that the moiety exists in at least a first state (adopted in the absence of activity against the target) and a second state (adopted in the presence of said activity). A determination of a change in state of the reporter moiety from first to second state therefore serves as a signal of activity against the target, which signal is acquired after microcompartmentalization of the microbeads and release of the chemical structures into solution (to yield TCSs for screening for activity against the target).

It will be appreciated that such a signal can be a positive signal (e.g. acquisition of fluorescence or a conformational shift) or a null signal (e.g. loss of fluorescence or absence of a conformational shift).

Thus, in the simplest case, the reporter moiety is the target itself (or a fragment thereof), and the assay system is for target binding activity. In such embodiments, the first state is unbound target, and the second state is a target-chemical compound complex. In this case, the assay system consists (or consists essentially of) the reporter moiety, since no other assay system components are involved in driving the change from first to second state. In such cases, the microcompartment need not contain additional target assay system component(s): the change in state of the reporter moiety can be detected by analysis of microbeads screened and released from opened microcompartment by physical techniques or via the use of suitable detection probes and/or reagents applied directly to the screened microbeads.

In another simple case, the reporter moiety is a substrate of an enzyme target, and the assay is for inhibitory activity against the enzyme target. In such embodiments, the first state is enzymatically modified substrate and the second state is non-enzymatically modified substrate. In this case, the assay system requires target enzyme as an additional component, and this is included in the microcompartment in which the microbead is assayed. The change in state of the reporter moiety can be detected by any convenient detection system, including antibody probes or gain (or loss) of fluorescence attendant on enzymatic modification, enzymatic incorporation of labelled markers etc. Thus, the change in state of the reporter moiety can again be detected by analysis of microbeads screened and released from opened microcompartment by physical techniques or via the use of suitable detection probes and/or reagents applied directly to the screened microbeads.

In yet another simple case, the reporter moiety is a binding partner (for example a ligand) for a receptor target, and the assay is for receptor blocking activity. In such embodiments, the first state is reporter moiety complexed with the receptor and the second state is uncomplexed reporter moiety. In this case, the assay system requires target receptor as an additional component, and this is included in the microcompartment in which the microbead is assayed. The change in state of the reporter moiety can be detected by any convenient detection system, including antibody probes or gain (or loss) of fluorescence attendant on ligand-receptor binding (for example by quenching of a fluorescent label on the reporter moiety by a quenching group on the receptor). Thus, the change in state of the reporter moiety can again be detected by analysis of microbeads screened and released from opened microcompartment by physical techniques or via the use of suitable detection probes and/or reagents applied directly to the screened microbeads.

Thus, those skilled in the art will appreciate that the system reporter moiety can be selected according to the nature of the target and the activity to be screened, and that the first and second states of the target assay may be distinguished on the basis of any modification which occurs during the change in state.

This can be detected by measurement of: (a) fluorescence, for example quenched or unquenched fluorescence; and/or (b) cleaved or uncleaved conformation; and/or (c) phosphorylated or non-phosphorylated states; (d) different glycosylation type, pattern or extent; and/or (e) different antigenic determinants; and/or (f) being bound or unbound to a ligand; and/or (g) being complexed or uncomplexed with one or more further assay system components.

This means that the microbeads of the invention can be used to encode information not only as to the nature of the chemical structures, but also of the activity of those chemical structures when assayed against a target in solution and free of any steric inhibition.

This greatly facilitates post-reaction screening, since the microbeads need not be maintained in a microcompartment after the state of the reporter moiety becomes fixed (and the signal is thereby generated), since a spatial association with the TCSs and/or other component(s) of the assay system is no longer required once the signal has been acquired.

The microbeads may therefore be removed, separated and/or isolated from the microcompartments in which the assay of the released TCSs is carried out and then subject to an extremely wide variety of physicochemical manipulations, including selection and fractionation techniques which would physically disrupt the relatively large and fragile microcompartments. This provides great flexibility, and can be exploited to greatly improve HTS throughput and hit deconvolution and characterization.

Such post assay reaction physicochemical manipulations include HTS protocols which include the use of FRET, FACS, immunoprecipitation, immunosedimentation, immunofiltration, centrifugal separation, gradient centrifugation, differential buoyancy separation, filtration, affinity column chromatography and/or magnetic microbead affinity selection high throughput screening. Thus the screening method of the invention may comprise FADS and/or FACS. The screening step may also comprise fluorescence analysis, including but not limited to FRET, FliM, fluorophore tagged antibody, fluorophore tagged DNA sequence or fluorescent dyes.

Chemical Structures

The chemical structures may be small molecules (as herein defined). In certain embodiments, the structures are comprised of a number of linked substructures. In preferred embodiments, the chemical structures are elaborated by split-and-pool methodologies (as described below).

In other embodiments, the chemical structures may be large molecules (as herein defined).

Split-and-Pool Generation of Chemical Structures

In preferred embodiments, a split-and-pool chemical structure/tagging technique is employed (see e.g. Mannocci et al. (2011) Chem. Commun., 47: 12747-12753, and in particular FIG. 3 thereof, which is hereby incorporated by reference).

In this approach, a core or series of cores are first immobilised onto the microbead surfaces. By varying the loading of the core at this stage, it is possible to control the quantity of compound that will ultimately be produced on the bead and thus allow for dose response determinations (which may be facilitated by incorporating information specifying the loading into the tag). In many embodiments, the loading of the chemical structures is selected such that the concentration after microcompartmentalization (for example, encapsulation within microdroplets) is within the range from pMolar to Molar concentrations. Thus, a plurality of pieces of DNA of the same sequence is added at this stage to encode the core structure as well as the loading of the core on the microbead. The encoding tag may therefore be present in multiple copies, and in some embodiments several million copies of the tag are immobilized on a single microbead.

Since the core has more than one vector on its surface it can be chemically modified by different chemical reactions to permit split and pool methods to be applied. In this methodology, the core is modified by addition of a large number of different chemical monomers to a specific vector, the only constraint being that each vector has to be modified by compatible chemistry. This can involve from 1 to 100,000 monomers. The nature of the monomer addition is encoded by ligating a new piece of DNA onto the DNA fragment already attached to the bead. The beads with the monomer are then pooled again into a single pool and then split into new populations and a second set of monomers added, this is also then encoded by DNA ligation. This can be repeated for any number of monomer additions. However, in a preferred methodology 2 or 3 vectors per core are used.

Clonal Tagging of Existing Chemical Libraries

Tagged chemical structures may be provided by any suitable means. For example, the microbead for use according to the invention may comprise a clonal population of chemical structures, and an encoding tag or tags may be releasably linked to the microbead. The clonal population of chemical structures in such embodiments may be an element of a commercially available chemical library.

Suitable nucleic acid-based tags are available commercially (e.g. from Twist Bioscience Corporation), but they may be synthesised as described in e.g. WO2015/021080 (the contents of which are hereby incorporated by reference).

Templated Synthesis of Chemical Structures

In certain embodiments, an encoding nucleic acid tag serves as a template for the chemical structure. In such embodiments, the library of tagged chemical structures is provided by nucleic acid-templated, for example DNA-templated, synthesis of the chemical structures followed by releasable linkage to the microparticle. Any suitable templating technology may be employed, and suitable techniques are described, for example, in Mannocci et al. (2011) Chem. Commun., 47: 12747-12753; Kleiner et al. (2011) Chem Soc Rev. 40(12): 5707-5717 and Mullard (2016) Nature 530: 367-369. Also suitable is the DNA-routing approach developed by Professor Pehr Harbury and co-workers (Stanford University, USA). The DNA template may be patterned/configured in any way: for example the YoctoReactor system employs three-way DNA-hairpin-looped junctions to assist the library synthesis by transferring appropriate donor chemical moieties onto a core acceptor site (see WO2006/048025, the disclosure of which is hereby incorporated by reference).

Alternative structural geometries are also available, such as 4-way DNA Holliday Junctions and hexagonal structures as described by Lundberg et al. (2008) Nucleic acids symposium (52): 683-684 and complex shapes and patterns created by the “scaffolded DNA origami” techniques reviewed by Rothemund (2006) Nature 440: 297-302.

Cleavable Linkers

The chemical structure(s) are releasably linked to the microbead by a cleavable linker.

Any cleavable linker may be used to releasably link the clonal population of chemical structure(s) to the microbead (and so, indirectly, to its encoding tag). In preferred embodiments, the cleavable linker is “scarless”. In such embodiments, the encoded chemical structure is released in a form in which it is completely or substantially free of linker residues, so that its activity in the screen is uncompromised by “scars” remaining after linker cleavage. It will be appreciated that some linker “scars” may be tolerated, such as —OH and/or —SH and/or —NH groups. The method of cleavage/cleaving agent is also preferably compatible with the assay system.

A wide range of suitable cleavable linkers are known to those skilled in the art, and suitable examples are described by Leriche et al. (2012) Bioorganic & Medicinal Chemistry 20 (2): 571-582. Suitable linkers therefore include enzymatically cleavable linkers; nucleophile/base-sensitive linkers; reduction sensitive linkers; photocleavable linkers; electrophile/acid-sensitive linkers; metal-assisted cleavage-sensitive linkers; oxidation-sensitive linkers; and combinations of two or more of the foregoing.

Enzyme cleavable linkers are described, for example, in: WO 2017/089894; WO 2016/146638; US2010273843; WO 2005/112919; WO 2017/089894; de Groot et al. (1999) J. Med. Chem. 42: 5277; de Groot et al. (2000) J Org. Chem. 43: 3093 (2000); de Groot et al., (2001) J Med. Chem. 66: 8815; WO 02/083180; Carl et al. (1981) J Med. Chem. Lett. 24: 479; Studer et al. (1992) Bioconjugate Chem. 3 (5): 424-429; Carl et al. (1981) J. Med. Chem. 24 (5): 479-480 and Dubowchik et al. (1998) Bioorg & Med. Chern. Lett. 8: 3347. They include linkers cleavable by enzymes selected from: proteases (including enterokinases), nucleases, nitroreductases, phosphatases, β-glucuronidase, lysosomal enzymes, TEV, trypsin, thrombin, cathepsin B, B and K, caspase, matrix metalloproteinases, phosphodiesterases, phospholipidases, esterases and β-galactosidases. Nucleophile/base cleavable linkers include: dialkyl dialkoxysilane, cyanoethyl group, sulfone, ethylene glycol disuccinate, 2-N-acryl nitrobenzenesulfonamide, α-thiophenylester, unsaturated vinyl sulphide, sulphonamide, malondialdehyde indole derivative, levulinoyl ester, hydrazine, acylhydrazone, alkyl thioester. Reduction cleavable linkers include disulphide bridges and azo compounds. Radiation cleavable linkers include: 2-Nitrobenzyl derivatives, phenacyl ester, 8-quinolinyl benzenesulphonate, coumarin, phosphotriester, bis-arylhydrazone, bimane bi-thiopropionic acid derivatives. Electrophilie/acid cleavable linkers include: paramethoxybenzyl derivatives, tert-butylcarbamate analogues, dialkyl or diaryl dialkoxysilane, orthoester, acetal, aconityl, hydrazine, β-thiopropionate, phosphoramadite, imine, trityl, vinyl ether, polyketal, alkyl 2-(diphenylphosphino) benzoate derivatives. Organometallic/metal catalysed cleavable linkers include: allyl esters, 8-hydroxylquinoline ester and picolinate ester. Linkers cleavable by oxidation include: vicinal diols and selenium compounds.

In certain embodiments, the cleavable linker comprises a combination of covalent and non-covalent bonds (for example hydrogen bonds arising from nucleic acid hybridization). The chemical structures may therefore be (directly or indirectly) releasably linked to the microparticle by nucleic acid hybridization. In such embodiments, the cleavable linker may comprise RNA and in such embodiments the cleaving agent may comprise an RNase. In other embodiments, the cleavable linker may comprise DNA and in such embodiments the cleaving agent may comprise a site-specific endonuclease. In cases where the cleavable linker arises from nucleic acid hybridization, the cleaving agent may comprise dehybridization, for example melting, of nucleic acid coupled to the chemical structure and hybridized to nucleic acid coupled to the microparticle.

In other embodiments, the cleavable linker may comprise a peptide and in such embodiments the cleaving agent may comprise a peptidase. Cleavable (for example enzyme cleavable) peptide linkers may contain a peptide moiety that consists of single amino acid, or a dipeptide or tripeptide sequence of amino acids. The amino acids may be selected from natural and non-natural amino acids, and in each case the side chain carbon atom may be in either D or L (R or S) configuration. Exemplary amino acids include alanine, 2-amino-2-cyclohexylacetic acid, 2-amino-2-phenylacetic acid, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, γ-aminobutyric acid, β,β-dimethyl γ-aminobutyric acid, α,α-dimethyl γ-aminobutyric acid, ornithine, and citrulline (Cit). Suitable amino acids also include protected forms of the foregoing amino acids in which the reactive functionality of the side chains is protected. Such protected amino acids include lysine protected with acetyl, formyl, triphenylmethyl (trityl), and monomethoxytrityl (MMT). Other protected amino acid units include arginine protected tosyl or nitro groups and ornithine-protected with acetyl or formyl groups.

Self-Immolative Linkers

Particularly suitable for use as cleavable linkers in the invention are self-immolative linkers comprising: (a) a cleavage moiety; and (b) a self-immolative moiety (“SIM”).

Such linkers may be used as shown diagrammatically in FIG. 1, which shows spontaneous elimination of the SIM following cleavage to release the free chemical structure.

Particularly suitable are self-immolative linkers comprising: (a) an enzymatic cleavage moiety; and (b) a SIM. In such embodiments, the enzymatic cleavage moiety may be a peptide sequence (cleavable with proteases) or a non-peptide enzymatically cleavable group, for example a glucuronide moiety incorporating a hydrophilic sugar group cleavable by beta-glucuronidase (as explained in McCombs and Owen (2015) Antibody Drug Conjugates: Design and Selection of Linker, Payload and Conjugation Chemistry The AAPS Journal 17(2): 339-351) and shown below:

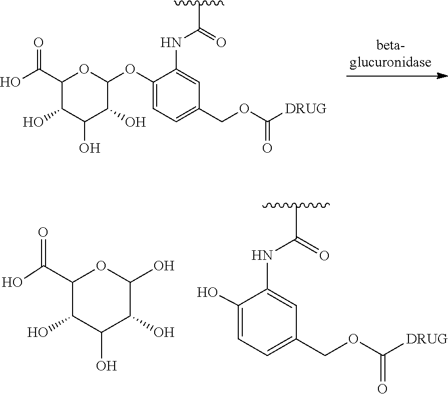

Suitable β-glucuronide-based linkers are described in WO 2007/011968, US 20170189542 and WO 2017/089894 (the contents of which are hereby incorporated by reference). Such linkers may therefore have the formula:

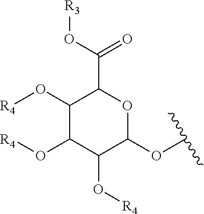

wherein R₃ is hydrogen or a carboxyl protecting group and each R₄ is independently hydrogen or a hydroxyl protecting group.

The SIM of the self-immolative linkers for use in the invention may be selected from a substituted alkyl, unsubstituted alkyl, substituted heteroalkyl, unsubstituted heteroalkyl, unsubstituted heterocycloalkyl, substituted heterocycloalkyl, substituted and unsubstituted aryl or substituted and unsubstituted heteroaryl. Suitable SIMs therefore include the p-aminobenzyl alcohol (PAB) unit and aromatic compounds that are electronically similar to the PAB group (such as the 2-aminoimidazol-5-methanol derivatives described by Hay et al. (1999) Bioorg. Med. Chem. Lett. 9: 2237) and ortho- or para-aminobenzylacetals.

Other suitable SIMs are those that undergo cyclization upon amide bond hydrolysis, for example the substituted and unsubstituted 4-aminobutyric acid amides described by Rodrigues et al. (1995) Chemistry Biology 2: 223). Yet further suitable SIMs include appropriately substituted bicyclo[2.2.1] and bicyclo[2.2.2] ring systems as described by Storm et al. (1972) J. Amer. Chem. Soc. 94: 5815 and various 2-aminophenylpropionic acid amides (see, e.g., Amsberry et al. (1990) J. Org. Chem. 55: 5867).

Particularly suitable are self-immolative peptide linkers comprising a peptide as cleavage moiety. FIGS. 2 and 3 show the construction and use of an encoded chemical library using such a linker. Here, the cleavable peptide is the dipeptide valine-citrulline and the SIM is p-aminobenzyl alcohol (PAB). In such embodiments, enzymatic cleavage of the amide-linked PAB triggers a 1,6-elimination of carbon dioxide and concomitant release of the free chemical structure. As also shown in FIG. 2, the encoding tag and chemical structure(s) may be linked via a bead, and a single bead may be loaded with a plurality (where n>1) of chemical structures, for example such that the ratio of encoding tag(s) to linked chemical structures is 1:10 to 1:1000. In such embodiments, the relatively small size of the peptide linker permits enhanced rates of diffusion and higher bead loadings, while the chemical structure needs only a single amine for functionalization.

Non-limiting examples of suitable cleavage moieties and SIMs for use as self-immolative linkers according to the invention are described, for example, in: WO 2017/089894; WO 2016/146638; US2010273843; WO 2005/112919; WO 2017/089894; de Groot et al. (1999) J. Med. Chem. 42: 5277; de Groot et al. (2000) J Org. Chem. 43: 3093 (2000); de Groot et al., (2001) J Med. Chem. 66: 8815; WO 02/083180; Carl et al. (1981) J Med. Chem. Lett. 24: 479; Studer et al. (1992) Bioconjugate Chem. 3 (5): 424-429; Carl et al. (1981) J. Med. Chem. 24 (5): 479-480 and Dubowchik et al. (1998) Bioorg & Med. Chern. Lett. 8: 3347 (the contents of which are hereby incorporated by reference).

Targets

Target Proteins

The target of the assay system of the invention may comprise a target protein. It may comprise an isolated target protein or isolated target protein complex. For example, the target protein/protein complex may be an intracellular target protein/protein complex. The target protein/protein complex may be in solution, or may be comprised a membrane or transmembrane protein/protein complex. In such embodiments, the chemical structures may be screened for ligands which bind to the target protein/protein complex. The ligands may be inhibitors of the target protein/protein complex.

It will be appreciated that any suitable target protein may be employed, including proteins from any of the target cells discussed in the following section. Thus, the target protein suitable for use in the assay systems according to the invention may be selected from eukaryotic, prokaryotic, fungal and viral proteins.

Suitable target proteins therefore include, but are not limited to, oncoproteins, transport (nuclear, carrier, ion, channel, electron, protein), behavioural, receptor, cell death, cell differentiation, cell surface, structural proteins, cell adhesion, cell communication, cell motility, enzymes, cellular function (helicase, biosynthesis, motor, antioxidant, catalytic, metabolic, proteolytic), membrane fusion, development, proteins regulating biological processes, proteins with signal transducer activity, receptor activity, isomerase activity, enzyme regulator activity, chaperone, chaperone regulator, binding activity, transcription regulator activity, translation regulator activity, structural molecule activity, ligase activity, extracellular organisation activity, kinase activity, biogenesis activity, ligase activity, and nucleic acid binding activity.

Target proteins may be selected from, and are therefore not limited to, DNA methyl transferases, AKT pathway proteins, MAPK/ERK pathway proteins, tyrosine kinases, epithelial growth factor receptors (EGFRs), fibroblast growth factor receptors (FGFRs), vascular endothelial growth factor receptors (VEGFRs), erythropoietin-producing human hepatocellular receptors (Ephs), tropomyosin receptor kinases, tumor necrosis factors, apoptosis regulator Bcl-2 family proteins, Aurora kinases, chromatin, G-protein coupled receptors (GPCRs), NF-κB pathway, HCV proteins, HIV proteins, Aspartyl proteases, Histone deacetylases (HDACs), glycosidases, lipases, histone acetyltransferase (HAT), cytokines and hormones.

Specific target proteins may be selected from ERK1/2, ERKS, A-Raf, B-Raf, C-Raf, c-Mos, Tpl2/Cot, MEK, MKK1, MKK2, MKK3, MKK4, MKK5, MKK6, MKK7, TYK2, JNK1, JNK2, JNK3, MEKK1, MEKK2, MEKK3, MEKK4, ASK1, ASK2, MLK1, MLK2, MLK3, p38 α, p38 β, p38 γ, p38 δ, BRD2, BRD3, BRD4, phosphatidyl inositol-3 kinase (PI3K), AKT, Protein kinase A (PKA), Protein Kinase B (PKB), Protein kinase C (PKC), PGC1α, SIRT1, PD-L1, mTOR, PDK-1, p70 S6 kinase, forkhead translocation factor, MELK, eIF4E, Hsp90, Hsp70, Hsp60, topoisomerase type I, topoisomerase type II, DNMT1, DNMT3A, DNMT3B, Cdk11, Cdk2, Cdk3, Cdk4, Cdk5, Cdk6, Cdk7, α-tubulin, β-tubulin, γ-tubulin, δ-tubulin, ε-Tubulin, Janus Kinases (JAK1, JAK2, JAK3), ABL1, ABL2, EGFR, EPH A1, EPHA2, EPHA3, EPHA4, EPHA5, EPHA6, EPHA7, EPHA8, EPHA10, EPHB1, EPHB2, EPHB3, EPHB4, EPHB6, HER2/neu, Her3, Her4, ALK, FGFR1, FGFR2, FGFR3, FGFR4, IGF1R, INSR, INSRR, VEGFR-1, VEGFR-2, VEGFR-3, FLT-3, FLT4, PDGFRA, PDGFRB, CSF1R, Axl, IRAK4, SCFR, Fyn, MuSK, Btk, CSK, PLK4, Fes, MER, c-MET, LMTK2, FRK, ILK, Lck, TIE1, FAK, PTK6, TNNI3, ROSCCK4, ZAP-70, c-Src, Tec, Lyn, TrkA, TrkB, TrkC, RET, ROR1, ROR2, ACK1, Syk, MDM2, HRas, KRas, NRas, ROCK, PI3K, BACE1, BACE2, CTSD, CTSE, NAPSA, PGC, Renin, MMSET, Aurora A kinase, Aurora B kinase, Aurora C kinase, farnesyltransferase, telomerase, adenylyly cyclase, cAMP phosphodiesterase, PARP1, PARP2, PARP4, PARP-5a, PARP-5b, PKM2, Keap1, Nrf2, TNF, TRAIL, OX40L Lymphotoxin-alpha, IFNAR1, IFNAR2, IFN-α, IFN-β, IFN-γ, IFNLR1, CCL3, CCL4, CCL5, IL1α, IL1β, IL-2, IL-2, IL-4, IL-5, IL-6, IL-7, IL-9, IL-9, IL-10, IL-11, IL-12, IL-13, IL-15, IL-17, Bcl-2, Bcl-xL, Bax, HCV helicase, E1, E2, p7, NS2, NS3, NS4A, NS4B, NS5A, NS5B, NF-κB1, NF-κB2, RelA, ReIB, c-Rel, RIP1, ACE, HIV protease, HIV integrase, Gag, Pol, gp160, Tat, Rev, Nef, Vpr, Vif, Vpu, RNA polymerase, GABA transaminase, Reverse transcriptase, DNA polymerase, prolactin, ACTH, ANP, insulin, PDE, AMPK, iNOS, HDAC1, HDAC2, HDAC3, HDAC4, HDAC5, HDAC6, HDAC7, HDAC8, HDAC9, HDAC10, HDAC11, lactase, amylase lysozyme, neuraminidase, invertase, chitinase, hyaluronidase, maltase, sucrase, phosphatase, phosphorylases, P, Histidine decarboxylase, PTEN, histone lysine demethylase (KDM), GCNS, PCAF, Hatt, ATF-2, Tip60, MOZ, MORF, HBO1, p300, CBP, SRC-1, SRC-3, ACTR, TIF-2, TAF1, TFIIIC, protein 0-mannosyl-transferase 1 (POMT1), amyloid β and Tau.

Target Cells

The invention finds particular application in phenotypic, cell-based assays since living or dead cells can be microcompartmentalized along with the microbeads of the invention. In such embodiments, the target assay system reporter moiety shifts state in response to changes in the target cell induced by chemical structures which exhibit a desired activity against that cell. Such changes include release of cytokines, metabolites, toxins, antibodies, hormones, signalling molecules or enzymes. In some embodiments, the assay system may be a homogeneous aqueous phase assay system, and may comprise a phenotypic screen. In such embodiments, the assay system may comprise a live target cell.

Any suitable target cell may be employed, as described below.

The target cell may be archaeal, for example selected from the phyla: (a) Crenarchaeota; (b) Euryarchaeota; (c) Korarchaeota; (d) Nanoarchaeota and (e) Thaumarchaeota, for example Haloferax volcanii or Sulfolobus spp.

Prokaryotic cells suitable for use as target cells according to the invention include bacterial cells. In such embodiments, the target cell may be a pathogenic bacterium. Other bacterial target cells include cells selected from Gram-positive bacteria (for example, selected from Enterococcus faecalis, Enterococcus faecium and Staphylococcus aureus); Gram-negative bacteria (for example, selected from Klebsiella pneumoniae, Acinetobacter baumanii, Escherichia coli, E. coli ST131 strains, Pseudomonas aeruginosa, Enterobacter cloacae, Enterobacter aerogenes and Neisseria gonorrhoeae) and bacteria exhibiting an indeterminate Gram reaction.

Eukaryotic cells suitable for use as target cells according to the invention include: (a) fungal; (b) mammalian; (c) a higher plant cell; (d) protozoal; (e) a helminth cell; (f) algal; (g) a cell derived from a clinical tissue sample, for example a human patient sample and (h) an invertebrate cell.

Suitable mammalian cells include cancer cells, for example human cancer cells, muscle cells, human neuronal cells and others cells derived from a living human patient that show a disease relevant phenotype.

In cases where the cell is a eukaryotic cell (for example a human cell), the cell may be selected from: totipotent, pluripotent, induced pluripotent, multipotent, oligopotent, stem, embryonic stem (ES), somatic, germ line, terminally differentiated, non-dividing (post-mitotic), mitotic, primary, cell-line-derived and tumour cells.

The cell is preferably isolated (i.e. not present in its natural cellular/tissue milieu) and/or metabolically active (for example being present in the assay system along with a culture or transport medium for maintaining cellular viability and/or activity and/or supporting cellular growth or proliferation).

Suitable eukaryotic cells may be isolated from an organism, for example in an organism selected from: metazoan, fungal (e.g. yeast), mammalian, non-mammalian, plant, protozoal, helminth, algal, insect (e.g. fly), fish (e.g. zebrafish), amphibian (e.g. frog), bird, invertebrate and vertebrate organisms.

Suitable eukaryotic cells may also be isolated from non-human animal selected from: mammal, rodent, rabbit, pig, sheep, goat, cow, rat, mouse, non-human primate and hamster. In other embodiments, the cell may be isolated from a non-human disease model or transgenic non-human animal expressing a heterologous gene, for example a heterologous gene encoding a therapeutic product.

Bacteria as Target Cells

The target cells for use according to the invention may be bacterial cells. In such embodiments, the bacteria may be selected from: (a) Gram-positive, Gram-negative and/or Gram-variable bacteria; (b) spore-forming bacteria; (c) non-spore forming bacteria; (d) filamentous bacteria; (e) intracellular bacteria; (f) obligate aerobes; (g) obligate anaerobes; (h) facultative anaerobes; (i) microaerophilic bacteria and/or (f) opportunistic bacterial pathogens.

In certain embodiments, target cells for use according to the invention may be selected from bacteria of the following genera: Acinetobacter (e.g. A. baumannii); Aeromonas (e.g. A. hydrophila); Bacillus (e.g. B. anthracis); Bacteroides (e.g. B. fragilis); Bordetella (e.g. B. pertussis); Borrelia (e.g. B. burgdorferi); Brucella (e.g. B. abortus, B. canis, B. melitensis and B. suis); Burkholderia (e.g. B. cepacia complex); Campylobacter (e.g. C. jejuni); Chlamydia (e.g. C. trachomatis, C. suis and C. muridarum); Chlamydophila (e.g. (e.g. C. pneumoniae, C. pecorum, C. psittaci, C. abortus, C. felis and C. caviae); Citrobacter (e.g. C. freundii); Clostridium (e.g. C. botulinum, C. difficile, C. perfringens and C. tetani); Corynebacterium (e.g. C. diphteriae and C. glutamicum); Enterobacter (e.g. E. cloacae and E. aerogenes); Enterococcus (e.g. E. faecalis and E. faecium); Escherichia (e.g. E. coli); Flavobacterium; Francisella (e.g. F. tularensis); Fusobacterium (e.g. F. necrophorum); Haemophilus (e.g. H. somnus, H. influenzae and H. parainfluenzae); Helicobacter (e.g. H. pylon); Klebsiella (e.g. K. oxytoca and K. pneumoniae), Legionella (e.g. L. pneumophila); Leptospira (e.g. L. interrogans); Listeria (e.g. L. monocytogenes); Moraxella (e.g. M. catarrhalis); Morganella (e.g. M. morganii); Mycobacterium (e.g. M. leprae and M. tuberculosis); Mycoplasma (e.g. M. pneumoniae); Neisseria (e.g. N. gonorrhoeae and N. meningitidis); Pasteurella (e.g. P. multocida); Peptostreptococcus; Prevotella; Proteus (e.g. P. mirabilis and P. vulgaris), Pseudomonas (e.g. P. aeruginosa); Rickettsia (e.g. R. rickettsii); Salmonella (e.g. serotypes. Typhi and Typhimurium); Serratia (e.g. S. marcesens); Shigella (e.g. S. flexnaria, S. dysenteriae and S. sonnei); Staphylococcus (e.g. S. aureus, S. haemolyticus, S. intermedius, S. epidermidis and S. saprophyticus); Stenotrophomonas (e.g. S. maltophila); Streptococcus (e.g. S. agalactiae, S. mutans, S. pneumoniae and S. pyogenes); Treponema (e.g. T. pallidum); Vibrio (e.g. V. cholerae) and Yersinia (e.g. Y. pestis).

The target cells for use according to the invention may be selected from high G+C Gram-positive bacteria and from low G+C Gram-positive bacteria.

Pathogenic Bacteria as Target Cells

Human or animal bacterial pathogens include such bacteria as Legionella spp., Listeria spp., Pseudomonas spp., Salmonella spp., Klebsiella spp., Hafnia spp, Haemophilus spp., Proteus spp., Serratia spp., Shigella spp., Vibrio spp., Bacillus spp., Campylobacter spp., Yersinia spp. Clostridium spp., Enterococcus spp., Neisseria spp., Streptococcus spp., Staphylococcus spp., Mycobacterium spp., Enterobacter spp.

Fungi as Target Cells

The target cells for use according to the invention may be fungal cells. These include yeasts, e.g. Candida species including C. albicans, C krusei and C tropicalis, and filamentous fungi such as Aspergillus spp. and Penicillium spp. and dermatophytes such as Trichophyton spp.

Plant Pathogens as Target Cells

The target cells for use according to the invention may be plant pathogens, for example Pseudomonas spp., Xylella spp., Ralstonia spp., Xanthomonas spp., Erwinia spp., Fusarium spp., Phytophthora spp., Botrytis spp., Leptosphaeria spp., powdery mildews (Ascomycota) and rusts (Basidiomycota).

Cancer Cells as Targets

Cancer cells may be used as target cells. Such cells may be derived from cell lines or from primary tumours. The cancer cells may be mammalian, and are preferably human. In certain embodiments, the cancer cells are selected from the group consisting of melanoma, lung, renal, colon, prostate, ovarian, breast, central nervous system and a leukaemic cell lines.

Suitable cancer cell lines include, without limitation, ovarian cancer cell lines (e.g. CaOV-3, OVCAR-3, ES-2, SK-OV-3, SW626, TOV-21G, TOV-112D, OV-90, MDA-H2774 and PA-I); breast cancer cell lines (e.g. MCF7, MDA-MB-231, MDA-MB-468, MDA-MB-361, MDA-MD-453, BT-474, Hs578T, HCC1008, HCC1954, HCC38, HCCI 143, HCCI 187, HCC1395, HCC1599, HCC1937, HCC2218, Hs574.T, Hs742.T, Hs605.T and Hs606); lung cancer cell lines (e.g. NCI-H2126, NCI-H1395, NCI-H1437, NCI-H2009, NCI-H1672, NCI-H2171, NCI-H2195, NCI-HI 184, NCI-H209, NCI-H2107 and NCI-H128); skin cancer cell lines (e.g. COL0829, TE354.T, Hs925.T, WM-115 and Hs688(A).T; bone cancer cell lines (e.g. Hs919.T, Hs821.T, Hs820.T, Hs704.T, Hs707(A).T, Hs735.T, Hs860.T, Hs888.T, Hs889.T, Hs890.T and Hs709.T); colon cancer cell lines (e.g. Caco-2, DLD-I, HCT-116, HT-29 and SW480); and gastric cancer cell lines (e.g. RF-I). Cancer cell lines useful in the methods of the present invention may be obtained from any convenient source, including the American Type Culture Collection (ATCC) and the National Cancer Institute.

Other cancer cell lines include those derived from neoplastic cells/subjects suffering from neoplasia, including proliferative disorders, benign, pre-cancerous and malignant neoplasia, hyperplasia, metaplasia and dysplasia. Proliferative disorders include, but are not limited to cancer, cancer metastasis, smooth muscle cell proliferation, systemic sclerosis, cirrhosis of the liver, adult respiratory distress syndrome, idiopathic cardiomyopathy, lupus erythematosus, retinopathy (e.g. diabetic retinopathy), cardiac hyperplasia, benign prostatic hyperplasia, ovarian cysts, pulmonary fibrosis, endometriosis, fibromatosis, harmatomas, lymphangiomatosis, sarcoidosis and desmoid tumours. Neoplasia involving smooth muscle cell proliferation include hyperproliferation of cells in the vasculature (e.g. intimal smooth muscle cell hyperplasia, restenosis and vascular occlusion, including in particular stenosis following biologically- or mechanically-mediated vascular injury, such as angioplasty). Moreover, intimal smooth muscle cell hyperplasia can include hyperplasia in smooth muscle other than the vasculature (e.g. blockage of the bile duct, bronchial airways and in the kidneys of patients with renal interstitial fibrosis). Non-cancerous proliferative disorders also include hyperproliferation of cells in the skin such as psoriasis and its varied clinical forms, Reiter's syndrome, pityriasis rubra pilaris and hyperproliferative variants of disorders of keratinization (including actinic keratosis, senile keratosis and scleroderma).

Cell Lines as Targets

Other cells derived from cell lines may be used as target cells. Such cells, which may preferably be human or mammalian, include those from patients suffering rare diseases with a detectable cellular phenotype. The cells may be of any type, including without limitation blood cells, immune cells, bone marrow cells, skin cells, nervous tissue and muscle cells.

Cell lines useful in the methods of the present invention may be obtained from any convenient source, including the American Type Culture Collection (ATCC) and the National Cancer Institute.

The cells/cell lines may, for example, be derived from subjects suffering from lysosomal storage diseases, muscular dystrophies, cystic fibrosis, Marfan syndrome, sickle cell anaemia, dwarfism, phenylketonuria, neurofibromatosis, Huntington disease, osteogenesis imperfecta, thalassemia and hemochromatosis.

The cells/cell lines may, for example, be derived from subjects suffering from other diseases including diseases and disorders of: blood, coagulation, cell proliferation and dysregulation, neoplasia (including cancer), inflammatory processes, immune system (including autoimmune diseases), metabolism, liver, kidney, musculoskeletal, neurological, neuronal and ocular tissues. Exemplary blood and coagulation diseases and disorders include: anaemia, bare lymphocyte syndrome, bleeding disorders, deficiencies of factor H, factor H-like 1, factor V, factor VIII, factor VII, factor X, factor XI, factor XII, factor XIIIA, factor XIIIB, Fanconi anaemia, haemophagocytic lymphohistiocytosis, haemophilia A, haemophilia B, haemorrhagic disorder, leukocyte deficiency, sickle cell anaemia and thalassemia.

Examples of immune related diseases and disorders include: AIDS; autoimmune lymphoproliferative syndrome; combined immunodeficiency; HIV-1; HIV susceptibility or infection; immunodeficiency and severe combined immunodeficiency (SCIDs). Autoimmune diseases which can be treated according to the invention include Grave's disease, rheumatoid arthritis, Hashimoto's thyroiditis, vitiligo, type I (early onset) diabetes, pernicious anaemia, multiple sclerosis, glomerulonephritis, systemic lupus E (SLE, lupus) and Sjogren syndrome. Other autoimmune diseases include scleroderma, psoriasis, ankylosing spondilitis, myasthenia gravis, pemphigus, polymyositis, dermomyositis, uveitis, Guillain-Barre syndrome, Crohn's disease and ulcerative colitis (frequently referred to collectively as inflammatory bowel disease (IBD)).

Other exemplary diseases include: amyloid neuropathy; amyloidosis; cystic fibrosis; lysosomal storage diseases; hepatic adenoma; hepatic failure; neurologic disorders; hepatic lipase deficiency; hepatoblastoma, cancer or carcinoma; medullary cystic kidney disease; phenylketonuria; polycystic kidney; or hepatic disease.

Exemplary musculoskeletal diseases and disorders include: muscular dystrophy (e.g. Duchenne and Becker muscular dystrophies), osteoporosis and muscular atrophy.

Exemplary neurological and neuronal diseases and disorders include: ALS, Alzheimer's disease; autism; fragile X syndrome, Huntington's disease, Parkinson's disease, Schizophrenia, secretase related disorders, trinucleotide repeat disorders, Kennedy's disease, Friedrich's ataxia, Machado-Joseph's disease, spinocerebellar ataxia, myotonic dystrophy and dentatorubral pallidoluysian atrophy (DRPLA).

Exemplary ocular diseases include: age related macular degeneration, corneal clouding and dystrophy, cornea plana congenital, glaucoma, leber congenital amaurosis and macular dystrophy.

The cells/cell lines may, for example, be derived from subjects suffering from diseases mediated, at least in part, by deficiencies in proteostasis, including aggregative and misfolding proteostatic diseases, including in particular neurodegenerative disorders (e.g. Parkinson's disease, Alzheimer's disease and Huntington's disease), lysosomal storage disorders, diabetes, emphysema, cancer and cystic fibrosis.

Archaea as Target Cells

The target cell may be archaeal, for example selected from the phyla: (a) Crenarchaeota; (b) Euryarchaeota; (c) Korarchaeota; (d) Nanoarchaeota and (e) Thaumarchaeota, for example Haloferax volcanii or Sulfolobus spp.

Exemplary, archaeal genera include Acidianus, Acidilobus, Acidococcus, Aciduliprofundum, Aeropyrum, Archaeoglobus, Bacilloviridae, Caldisphaera, Caldivirga, Caldococus, Cenarchaeum, Desulfurococcus, Ferroglobus, Ferroplasma, Geogemma, Geoglobus, Haladaptaus, Halalkalicoccus, Haloalcalophilium, Haloarcula, Halobacterium, Halobaculum, Halobiforma, Halococcus, Haloferax, Halogeometricum, Halomicrobium, Halopiger, Haloplanus, Haloquadratum, Halorhabdus, Halorubrum, Halosarcina, Halosimplex, Halostagnicola, Haloterrigena, Halovivax, Hyperthermus, lgnicoccus, lgnisphaera, Metallosphaera, Methanimicrococcus, Methanobacterium, Methanobrevibacter, Methanocalculus, Methantxaldococcus, Methanocella, Methanococcoides, Methanococcus, Methanocorpusculum, Methanoculleus, Methanofollis, Methanogenium, Methanohalobium, Methanohalophilus, Methanolacinia, Methanolobus, Methanomethylovorans, Methanomicrobium, Methanoplanus, Methanopyrus, Methanoregula, Methanosaeta, Methanosalsum, Methanosarcina, Methanosphaera, Melthanospirillum, Methanothermobacter, Methanothermococcus, Methanothermus, Methanothrix, Methanotorris, Nanoarchaeum, Natrialba, Natrinema, Natronobacterium, Natronococcus, Natronolimnobius, Natronomonas, Natronorubrum, Nitracopumilus, Palaeococcus, Picrophilus, Pyrobaculum, Pyrococcus, Pyrodictium, Pyrolobus, Staphylothermus, Stetteria, Stygiolobus, Sulfolobus, Sulfophobococcus, Sulfurisphaera, Thermocladium, Thermococcus, Thermodiscus, Thermofilum, Thermoplasma, Thermoproteus, Thermosphaera and Vulcanisaeta.

Exemplary archaeal species include: Aeropyrum pernix, Archaeglobus fulgidus, Archaeoglobus fulgidus, Desulforcoccus species TOK, Methanobacterium thermoantorophicum, Methanococcus jannaschii, Pyrobaculum aerophilum, Pyrobaculum calidifontis, Pyrobaculum islandicum, Pyrococcus abyssi, Pyrococcus GB-D, Pyrococcus glycovorans, Pyrococcus horikoshii, Pyrococcus spp. GE23, Pyrococcus spp. ST700, Pyrococcus woesii, Pyrodictium occultum, Sulfolobus acidocaldarium, Sulfolobus solataricus, Sulfolobus tokodalii, Thermococcus aggregans, Thermococcus barossii, Thermococcus celer, Thermococcus fumicolans, Thermococcus gorgonarius, Thermococcus hydrothermalis, Thermococcus onnurineus NA1, Thermococcus pacificus, Thermococcus profundus, Thermococcus siculi, Thermococcus spp. GE8, Thermococcus spp. JDF-3, Thermococcus spp. TY. Thermococcus thioreducens, Thermococcus zilligti, Thermoplasma acidophilum, Thermoplasma volcanium, Acidianus hospitalis, Acidilobus sacharovorans, Aciduliprofundum boonei, Aeropyrum pernix, Archaeoglobus fulgidus, Archaeoglobus profundus, Archaeoglobus veneficus, Caldivirga maquilingensis, Candidatus Korarchaeum cryptofilum, Candidatus Methanoregula boonei, Candidatus Nitrosoarchaeum limnia, Cenarchaeum symbiosum, Desulfurococcus kamchatkensis, Ferroglobus placidus, Ferroplasma acidarmanus, Halalkalicoccus jeotgali, Haloarcula hispanica, Holaoarcula marismortui, Halobacterium salinarum, Halobacterium species, Halobiforma lucisalsi, Haloferax volvanii, Halogeometricum borinquense, Halomicrobium mukohataei, halophilic archaceon sp. DL31, Halopiger xanaduensis, Haloquadratum walsbyi, Halorhabdus tiamatea, Halorhabdus utahensis, Halorubrum lacusprofundi, Haloterrigena turkmenica, Hyperthermus butylicus, lgniococcus hospitalis, lgnisphaera aggregans, Metallosphaera cuprina, Metallosphaera sedula, Methanobacterium sp. AL-21, Methanobacterium sp. SWAN-1, Methanobacterium thermoautrophicum, Methanobrevibacter ruminantium, Methanobrevibacter smithii, Methanocaldococcus fervens, Methanocaldococcus infernus, Methanocaldococcus jannaschii, Methanocaldococcus sp. FS406-22, Methanocaldococcus vulcanius, Methanocella conradii, Methanocella paludicola, Methanocella sp. Rice Cluster I (RC-I). Methanococcoides burtonii, Methanococcus aeolicus, Methanococcus maripaludis, Methanococcus vannielii, Methanococcus voltae, Methanocorpusculum labreantum, Methanoculleus marisnigri, Methanohalobium evestigatum, Methanohalophilus Methanoplanus petrolearius, Methanopyrus kandleri, Methanosaeta concilii, Methanosaeta harundinacea, Methanosaeta thermophila, Methanosalsum zhilinae, Methanosarcina acetivorans, Methanosarcina barkeri, Methanosarcina mazei, Methanosphaera stadtmanae, Methanosphaerula palustris, Methanospiriullum hungatei, Mathanothermobacter marburgensis, Methanothermococcus okinawensis, Methanothermus fervidus, Methanotorris igneus, Nanoarchaeum equitans, Natrialba asiatica, Natrialba magadii, Natronomonas pharaonis, Nitrosopumilus maritimus, Picrophilus torridus, Pyrobaculum aerophilum, Pyrobaculum arsenaticum, Pyrobaculum calidifontis, Pyrobaculum islandicum, Pyrobaculum sp. 1860, Pyrococcus abyssi, Pyrococcus furiosus, Pyrococcus horikoshii, Pyrococcus sp. NA42, Pyrococcus yayanosii, Pyrolobus fumarii, Staphylothermus hellenicus, Staphylothermus marinus, Sulfolobus acidocaldirius, Sulfolobus islandicus, Sulfolobus solfataricus, Sulfolobus tokodaii, Thermococcus barophilus, Thermococcus gammatolerans, Thermococcus kodakaraensis, Thermococcus litoralis, Thermococcus onnurineus, Thermococcus sibiricus, Thermococcus sp. 4557, Thermococcus sp. AM4, Thermofilum pendens, Thermoplasma acidophilum, Thermoplasma volcanium, Thermoproteus neutrophilus, Thermoproteus tenax, Thermoproteus uzoniensis, Thermosphaera aggregans, Vulcanisaeta distributa, and Vulcanisaeta moutnovskia.

Particular examples of archaeal cells useful as producer cells according to the invention include Haloferax volcanii and Sulfolobus spp.

Library Microcompartments

The microbeads of the invention may be used in HTS when microcompartmentalized. The library microcompartments may take any form, provided that spatial association of TCSs released from the microbead is maintained i.e. micro-compartmentalization must be achieved such that the TCS and its cognate microbead from which it was released are confined in spatial proximity.

The chemical structures may be present in the library microcompartment(s) at a concentration sufficiently high as to permit cell-based or phenotypic screens, particularly homogeneous cell-based phenotypic assays. In certain embodiments, the tagged chemical structures may be present in the library microcompartment(s) at a concentration of at least: 0.1 nM, 0.5 nM, 1.0 nM 5.0 nM, 10.0 nM, 15.0 nM, 20.0 nM, 30.0 nM, 50.0 nM, 75.0 nM, 0.1 μM, 0.5 μM, 1.0 μM, 5.0 μM, 10.0 μM, 15.0 μM, 20.0 μM, 30.0 μM, 50.0 μM, 75.0 μM, 100.0 μM, 200.0 μM, 300.0 μM, 500.0 μM, 1 mM, 2 mM, 5 mM or 10 mM.

In other embodiments, the chemical structures may be present in the library microcompartment(s) at a concentration of at least: 0.1 μM, 0.5 μM, 1.0 μM 5.0 μM, 10.0 μM, 15.0 μM, 20.0 μM, 30.0 μM, 50.0 μM, 75.0 μM 0.1 nM, 0.5 nM, 1.0 nM 5.0 nM, 10.0 nM, 15.0 nM, 20.0 nM, 30.0 nM, 50.0 nM, 75.0 nM, 0.1 μM, 0.5 μM, 1.0 μM, 5.0 μM, 10.0 μM, 15.0 μM, 20.0 μM, 30.0 μM, 50.0 μM, 75.0 μM, 100.0 μM, 200.0 μM, 300.0 μM, 500.0 μM, 1 mM, 2 mM, 5 mM or 10 mM.

In other embodiments, the chemical structures may be present in the library microcompartments(s) at a concentration of: less than 1 μM; 1-100 μM; greater than 100 μM; 5-50 μM or 10-20 μM.

The library microcompartments contain the microbead of the invention and an aqueous solvent. The microcompartments may also contain other components, such as a cleaving agent for releasing the chemical structure(s) from the microbead into solution and/or one or more additional component(s) of said target assay system. In embodiments where the target is a cell, the library microcompartments may also contain one or more of the target cells.

The methods of the invention involve the step of releasing each chemical structure from its microbead to produce a plurality of free, tagless chemical structures (TCSs). The TCSs are conveniently released into the library microcompartments by diffusion, for example by diffusion after solvation.

Physical confinement can be achieved through the use of various micro-compartments, including microdroplets, microparticles and microvesicles. Preferred are microdroplets, as described in more detail in the following sections.

Microdroplets

Suitable materials and methods for preparing and processing microdroplets suitable for use in micro-compartmentalization according to the invention form part of the common general knowledge of those skilled in the art, being described, for example, in WO2010/009365, WO2006/040551, WO2006/040554, WO2004/002627, WO2004/091763, WO2005/021151, WO2006/096571, WO2007/089541, WO2007/081385 and WO2008/063227 (the contents of which are hereby incorporated by reference).

The size of the microdroplets will be selected by reference to the nature of the chemical structures and assay system to be encapsulated. The microdroplets may be substantially spherical with a diameter of: (a) less than 1 μm; (b) less than 10 μm; (c) 0.1-10 μm; (d) 10 μm to 500 μm; (b) 10 μm to 200 μm; (c) 10 μm to 150 μm; (d) 10 μm to 100 μm; (e) 10 μm to 50 μm; or (f) about 100 μm.

The microdroplets are preferably uniform in size such that the diameter of any droplet within the library will vary less than 5%, 4%, 3%, 2%, 1% or 0.5% when compared to the diameter of other droplets within the same library. In some embodiments, the microdroplets are monodisperse. However, polydisperse microdroplets may also be used according to the invention.

In single W/O type emulsions, the carrier liquid may be any water-immiscible liquid, for example an oil, optionally selected from: (a) a hydrocarbon oil; (b) a fluorocarbon oil; (c) an ester oil; (d) a silicone oil; (e) an oil having low solubility for biological components of the aqueous phase; (f) an oil which inhibits molecular diffusion between microdroplets; (g) an oil which is hydrophobic and lipophobic; (h) an oil having good solubility for gases; and/or (i) combinations of any two or more of the foregoing.

Thus, the microdroplets may be comprised in a W/O emulsion wherein the microdroplets constitute an aqueous, dispersed, phase and the carrier liquid constitutes a continuous oil phase.

In other embodiments, the microdroplets are comprised in a W/O/W double emulsion and the carrier liquid may an aqueous liquid. In such embodiments, the aqueous liquid may be phosphate buffered saline (PBS).

The microdroplets may therefore be comprised in a W/O/W double emulsion wherein the microdroplets comprise: (a) an inner core of aqueous growth media enveloped in an outer oil shell as the dispersed phase, and (b) the carrier liquid as the continuous aqueous phase. It will of course be appreciated that O/W/O droplets may be particularly useful for screening non-biological entities.

Surfactants for Use in Microdroplets

In embodiments where the microdroplets are comprised in an emulsion, the carrier liquid may constitute the continuous phase and the microdroplets the dispersed phase, and in such embodiments the emulsion may further comprise a surfactant and optionally a co-surfactant.

The surfactant and/or co-surfactant may be located at the interface of the dispersed and continuous phases, and when the microdroplets are comprised in a W/O/W double emulsion the surfactant and/or co-surfactant may be located at the interface of aqueous core and oil shell and at the interface of the oil shell and outer continuous phase.

A wide range of suitable surfactants are available, and those skilled in the art will be able to select an appropriate surfactant (and co-surfactant, if necessary) according to the selected screening parameters. For example, suitable surfactants are described in Bernath et al. (2004) Analytical Biochemistry 325: 151-157; Holtze and Weitz (2008) Lab Chip 8(10): 1632-1639; and Holtze et al. (2008) Lab Chip. 8(10):1632-1639. Other suitable surfactants, including fluorosurfactants in particular, are described in WO2010/009365 and WO2008/021123 (the contents of which are hereby incorporated by reference).

The surfactant(s) and/or co-surfactant(s) are preferably incorporated into the W/O interface(s), so that in embodiments where single W/O type emulsions are used the surfactant(s) and or co-surfactant(s) may be present in at the interface of the aqueous growth medium microdroplets and the continuous (e.g., oil) phase. Similarly, where double W/O/W type emulsions are used for co-encapsulation according to the invention, the surfactant(s) and or co-surfactant(s) may be present at either or both of the interfaces of the aqueous core and the immiscible (e.g. oil) shell and the interface between the oil shell and the continuous aqueous phase.

The surfactant(s) are preferably biocompatible. For example, the surfactant(s) may be selected to be non-toxic to any cells used in the screen. The selected surfactant(s) may also have good solubility for gases, which may be necessary for the growth and/or viability of any encapsulated cells.

Biocompatibility may be determined by any suitable assay, including assays based on tests for compatibility with a reference sensitive biochemical assay (such as in vitro translation) which serves as a surrogate for biocompatibility at the cellular level. For example, in vitro translation (IVT) of plasmid DNA encoding the enzyme β-galactosidase with a fluorogenic substrate (fluorescein di-β-D-galactopyranoside (FDG)) may be used as an indicator of biocompatibility since a fluorescent product is formed when the encapsulated DNA, the molecules involved in transcription and translation, and the translated protein do not adsorb to the drop interface and the higher-order structure of the protein remains intact.

Biocompatibility may also be determined by growing cells to be used in an assay in the presence of the surfactant and staining the cell with antibodies or viable cell dyes and determining the overall viability for the cell population compared to a control in the absence of the surfactant.

The surfactant(s) may also prevent the adsorption of biomolecules at the microdroplet interface. The surfactant may also function to isolate the individual microdroplets (and the corresponding microcultures). The surfactant preferably stabilizes (i.e. prevents coalescence) of the microdroplets. Stabilization performance can be monitored by e.g. phase-contrast microscopy, light scattering, focused beam reflectance measurement, centrifugation and/or rheology.

The surfactant may also form a functional part of the assay system, and may for example act to partition or sequester reactants and/or analytes and/or other moieties present in the assay (such as released tags) from other components. For example, a nickel complex in a hydrophilic head group of a functional surfactant can concentrate histidine-tagged proteins at the surface (see e.g. Kreutz et al. (2009) J Am Chem Soc. 131(17): 6042-6043). Such functionalized surfactants may also act as catalysts for small molecule synthesis (see e.g. Theberge et al. (2009) Chem. Commun.: 6225-6227). They may also be used to cause cell lysis (see e.g. Clausell-Tormos et al. (2008) Chem Biol. 15(5): 427-37). The present invention therefore contemplates the use of such functionalized surfactants.

Oils for Use in Emulsion Co-Encapsulation

While it will be appreciated than any liquid immiscible with the discontinuous phase may be used in the formation of microdroplet emulsions for use according to the invention, the immiscible fluid is typically an oil.

Preferably, an oil is selected having low solubility for biological components of the aqueous phase. Other preferred functional properties include tunable (e.g. high or low) solubility for gases, the ability to inhibit molecular diffusion between microdroplets and/or combined hydrophobicity and lipophobicity. The oil may be a hydrocarbon oil, for example light mineral oils, fluorocarbon oils, silicone oils or ester oils. Mixtures of two or more of the above-described oils are also preferred.

Examples of suitable oils are described in WO2010/009365, WO2006/040551, WO2006/040554, WO2004/002627, WO2004/091763, WO2005/021151, WO2006/096571, WO2007/089541, WO2007/081385 and WO2008/063227 (the contents of which are hereby incorporated by reference).

Processes for Microdroplet Emulsification

A wide range of different emulsification methods are known to those skilled in the art, any of which may be used to create the microdroplets of the invention.

Many emulsification techniques involve mixing two liquids in bulk processes, often using turbulence to enhance drop breakup. Such methods include vortexing, sonication, homogenization or combinations thereof.

In these “top-down” approaches to emulsification, little control over the formation of individual droplets is available, and a broad distribution of microdroplet sizes is typically produced. Alternative “bottom up” approaches operate at the level of individual drops, and may involve the use of microfluidic devices. For example, emulsions can be formed in a microfluidic device by colliding an oil stream and a water stream at a T-shaped junction: the resulting microdroplets vary in size depending on the flow rate in each stream.

A preferred process for producing microdroplets for use according to the invention comprises flow focusing (as described in e.g. Anna et al. (2003) Appl. Phys. Lett. 82(3): 364-366). Here, a continuous phase fluid (focusing or sheath fluid) flanking or surrounding the dispersed phase (focused or core fluid), produces droplet break-off in the vicinity of an orifice through which both fluids are extruded. A flow focusing device consists of a pressure chamber pressurized with a continuous focusing fluid supply. Inside, one or more focused fluids are injected through a capillary feed tube whose extremity opens up in front of a small orifice linking the pressure chamber with the external ambient environment. The focusing fluid stream moulds the fluid meniscus into a cusp giving rise to a steady micro or nano-jet exiting the chamber through the orifice; the jet size is much smaller than the exit orifice. Capillary instability breaks up the steady jet into homogeneous droplets or bubbles.

The feed tube may be composed of two or more concentric needles and different immiscible liquids or gases be injected leading to compound drops. Flow focusing ensures an extremely fast as well as controlled production of up to millions of droplets per second as the jet breaks up.

Other microfluidic processing techniques include pico-injection, a technique in which reagents are injected into aqueous drops using an electric field (see e.g. Eastburn et al. (2013) Picoinjection Enables Digital Detection of RNA with Droplet RT-PCR. PLoS ONE 8(4): e62961. doi:10.1371/journal.pone.0062961). Microdroplets can be fused to bring two reagents together, for example actively by electrofusion (see e.g. Tan and Takeuchi (2006) Lab Chip. 6(6): 757-63) or passively (as reviewed by Simon and Lee (2012) “Microdroplet Technology”, in Integrated Analytical Systems pp 23-50, 10.1007/978-1-4614-3265-4_2).

In all cases, the performance of the selected microdroplet forming process may be monitored by phase-contrast microscopy, light scattering, focused beam reflectance measurement, centrifugation and/or rheology.

Fluorescence-Activated Microdroplet Sorting

As explained herein, the methods of invention are suitable for high-throughput screening, since they involve microcompartmentalizing the microbeads in tiny volumes of solvent in the form of discrete microdroplets. This permits each microdroplet to be treated as a separate culture vessel, permitting rapid screening of large numbers of individual liquid co-cultures using established microfluidic and/or cell-sorting methodologies.

Microdroplets may be sorted by adapting well-established fluorescence-activated cell sorting (FACS) devices and protocols. This technique has been termed Fluorescence-Activated Droplet Sorting (FADS), and is described, for example, in Baret et al. (2009) Lab Chip 9: 1850-1858. Any change that can be detected by using a fluorescent moiety can be screened for.

As explained above, the change in state of the reporter moiety immobilized on the microbead contained within the microdroplet may be a fluorescent signal. This permits application of FADS. A variety of fluorescent proteins can be used as labels for this purpose, including for example the wild type green fluorescent protein (GFP) of Aequorea victoria (Chalfie et al. 1994, Science 263:802-805), and modified GFPs (Heim et al. 1995, Nature 373:663-4; PCT publication WO 96/23810). Alternatively, DNA2.0's IP-Free© synthetic non-Aequorea fluorescent proteins can be used as a source of different fluorescent protein coding sequences that can be amplified by PCR or easily excised using the flanking BsaI restriction sites and cloned into any other expression vector of choice.

Transcription and translation of this type of reporter gene leads to accumulation of the fluorescent protein in the cells, so rendering them amenable to FADS.

Alternatively, a huge range of dyes are available that fluoresce at specific levels and conditions within a cell. Examples include those available from Molecular probes (Thermo Scientific). Alternatively cellular components can be detected with antibodies and these can be stained with any number of fluorophores using commercially available kits. Similarly DNA sequences can be introduced labelled with fluorophores into a cell and will adhere by hybridisation to complementary DNA and RNA sequences in the cell allowing direct detection of gene expression in a process called Fluorescent in-situ hybridisation (FISH).

Any labelling process that can be applied in current high content screening can be applied to FADS and be detected as a change in fluorescent signal relative to controls.

Encoded Chemical Libraries (ECLs)

The screening methods of the invention are applied to an encoded chemical library (ECL) comprising a plurality of microcompartments of the invention, wherein each of the microcompartments contains a different chemical structure.

The ECL preferably comprises a number n of clonal populations of chemical structures, each clonal population being confined to n discrete library microcompartments. In such embodiments: (a) n>10³; or (b) n>10⁴; or (c) n>10⁵; or (d) n>10⁶; or (e) n>10⁷; or (f) n>10⁸; or (g) n>10⁹; or (h) n>10¹⁰; or (i) n>10¹¹; (j) n>10¹²; (k) n>10¹³; (l) n>10¹⁴; or (m) n>10¹⁵. Particularly preferred are ECLs wherein n=10⁶ to 10⁹.

Sequence-Based Structure-Activity Analysis

The screening methods of the invention comprises the step of screening the released microbeads by determining the state of the reporter moieties, whereby chemical structures having activity against the target can be identified by decoding the tags of microbeads having reporter moieties in the first state.

In cases where the tag comprises a nucleic acid sequence, the decoding step may comprise sequencing the nucleic acid. In such embodiments, the method may further comprise comparing the sequences of a plurality of different screened TCSs. Such a step may be followed by a step of performing sequence activity relationship analysis on the screened TCSs, which can permit classification of screened library members into different chemotypes.

### EXEMPLIFICATION

The invention will now be described with reference to specific Examples. These are merely exemplary and for illustrative purposes only: they are not intended to be limiting in any way to the scope of the monopoly claimed or to the invention described.

### FIGURE LEGENDS

FIG. 1: Schematic representation of release of free chemical structure using a self-immolative linker.

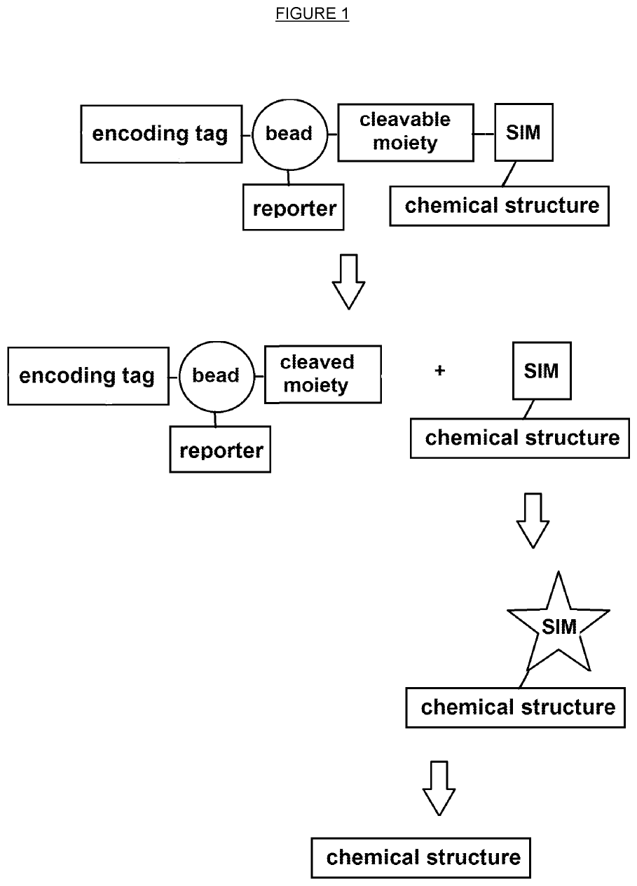

FIG. 2: Schematic representation of split-and-pool DECL using a self-immolative dipeptide linker.

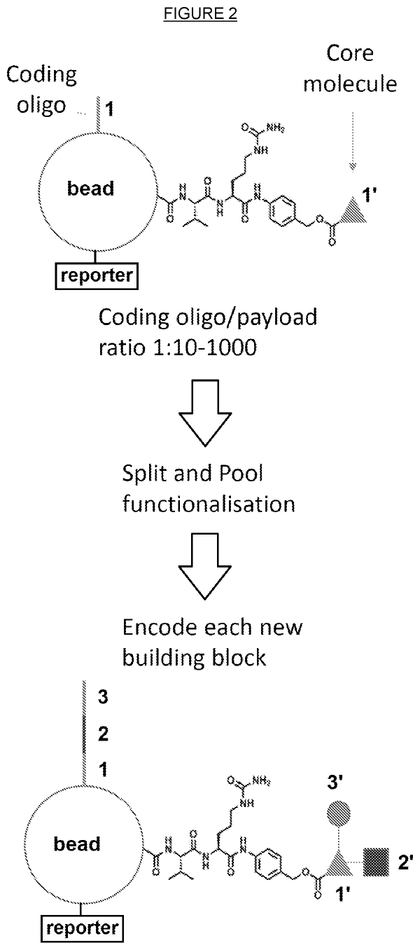

FIG. 3: Schematic representation of release of free chemical structures using a Val-Cit-PAB self-immolative peptide linker.

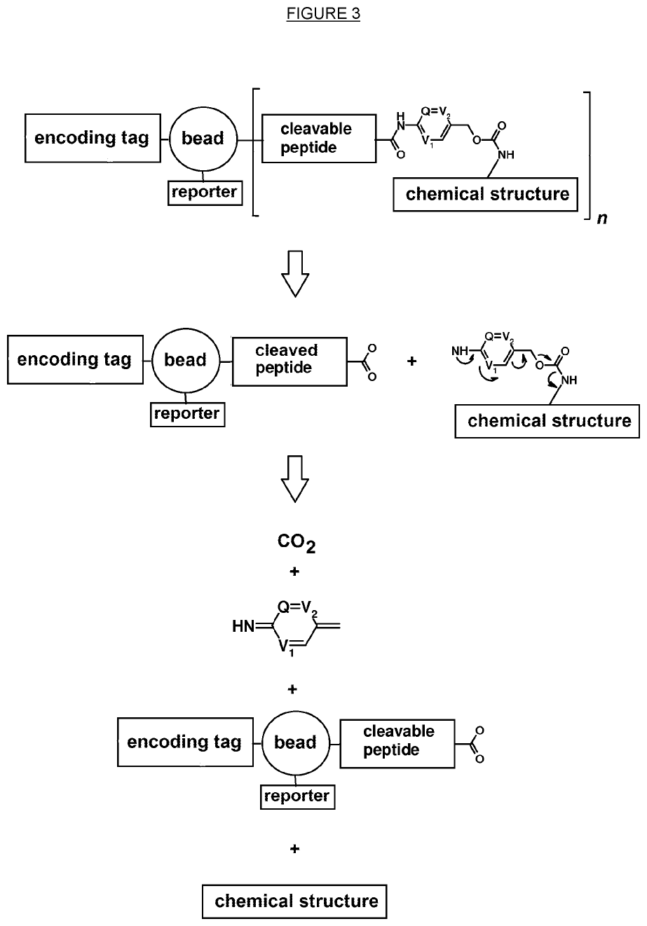

FIG. 4: A self-immolative process showing decrease of substrate A and increase of product B.

FIG. 5: Proportional compound loading on beads, showing clear distinct populations for each of loading sample.

FIG. 6: Schematic representation of enzyme activity measurement in micro-droplets using fluorescent reporter moiety.

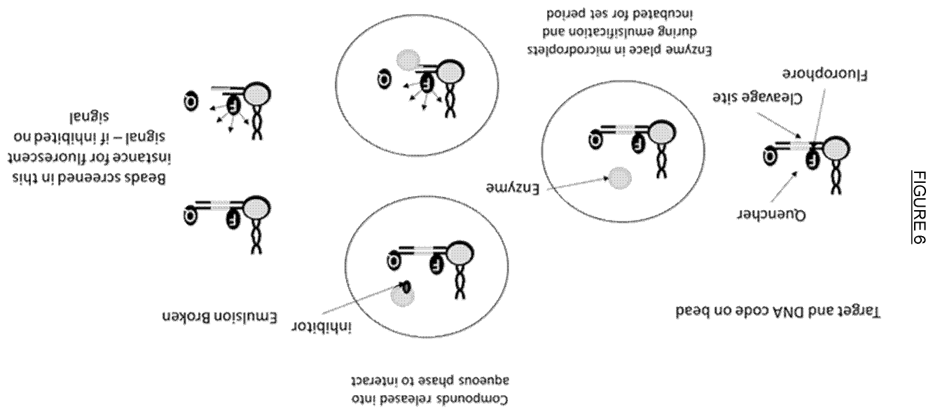

FIG. 7: Relative intensity of FITC signal in micro-droplets using fluorescent reporter moiety.

### EXAMPLE 1: ‘SCAR-LESS’ RELEASE OF A SMALL MOLECULE COMPOUND FROM A BEAD-LINKAGE-COMPATIBLE LINKER

Stock solutions of test substrate A was made up at 10 mg/ml in DMSO and NADPH (Sigma Aldrich) at 10 mg/ml (11.9 mM) in 40 mM MOPS, pH 7.5, 150 mM NaCl.

20 μl substrate A solution was combined with 480p1 of 40 mM MOPS pH 7.5 150 mM NaCl buffer and 500 μL NADPH solution. Reaction was initiated by adding 3 μL (29.6 mg/ml) nitroreductase (Prozomix) and incubated for 20 minutes at room temperature. 50 μl reaction solution was combined with 200 μl 3:1 ACN: H₂O+0.1% formic acid run on an LCMS (Agilent Technologies, Infinity 1290).

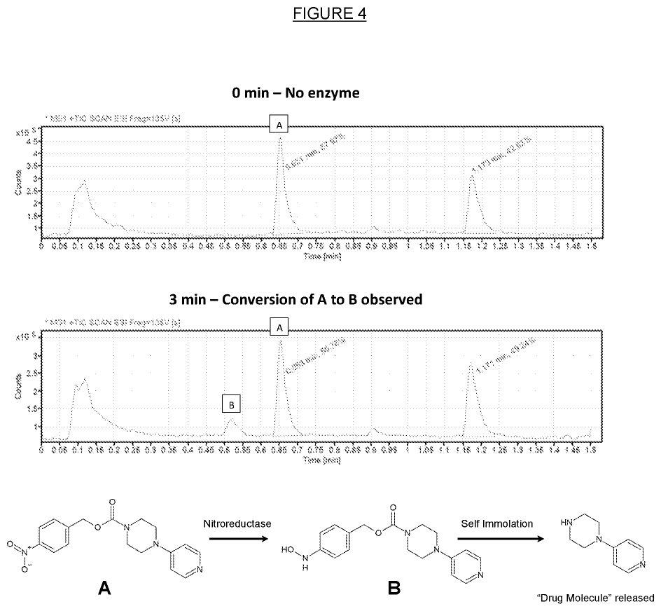

A clear decrease of substrate A and increase of product B could be observed (see FIG. 4) demonstrating a self-immolative process where the desired ‘drug molecule’ was released at 3 minutes. This result demonstrates controllable enzymic cleavage of a useful and specific linker resulting in ‘scar-less’ release of a small molecule. This linker is therefore useful for releasable attachment of small molecules to beads for use in activity assays.

### EXAMPLE 2: ACCURATE PROPORTIONAL COMPOUND LOADING ON BEADS

Preparation

50 ml of a 100 μM stock was made of 4-(aminomethyl) fluorescein hydrochloride (AMF.HCl) solution using 1.99 mg of AMF.HCl in 50 ml of 100 mM MOPS, 150 mM NaCl. This solution was diluted using 100 mM MOPS pH 7.5, 150 mM NaCl to 10 μM and 3 μM as required.

The following solutions were placed into 5×50 ml falcon tubes (1 mL each) for loading test.

In all cases DMTMM (4-(4,6-Dimethoxy-1,3,5-triazin-2-yl)-4-methylmorpholinium chloride, Sigma Aldrich) was used as a coupling reagent (assuming 10% of all acids on bead)=4.77×10−7 mol—Volume of 4.8 mM stock with 100 μl added to each falcon:

Falcon 1-0.11% loading—5.247×10⁻⁹ mol of AMF required—Volume of 100 μM (AMF.HCl) (Fisher) stock used=52.5 μL;

Falcon 2-0.033% loading—1.574×10⁻⁹ mol of AMF required—Volume of 100 μM AMF.HCl stock used=15.7 μL;

Falcon 3-0.011% loading—5.247×10⁻¹° mol of AMF required—Volume of 10 μM AMF.HCl stock used=52.5 μL;

Falcon 4-0.0033% loading—1.574×10⁻¹° mol of AMF required—Volume of 3 μM AMF.HCl stock used=15.7 μL;

Falcon 5-0.0011% loading—5.247×10⁻¹¹ mol of AMF required—Volume of 10 μM AMF.HCl stock used=5.25 μL.

Procedure

3.9 ml 100 mM MOPS pH 7.5, 150 mM NaCl was added to 5×50 mL falcons. 100 μL of freshly made 4.8 mM DMTMM stock solution was added and mixed thoroughly. 1 ml of 50 μm bead suspension was added (35 beads/μl) to each falcon and mixed for 60 minutes. In five separate 50 mL falcons AMF.HCl was added as shown above.

Beads were reacted with DMTMM for 60 mins, then poured into associated AMF 50 mL falcon and reacted overnight at room temperature.

Beads were pelleted at 1000 g for 10 minutes and as much solvent as possible was removed. 15 ml fresh buffer was added, mixed and wash steps repeated a further 2 times. Bead samples were analysed using FACS (MoFloXDP, Beckmann Coulter) exciting at Excite AFM on the beads using 488 nM laser and measuring emission using a 540 nm+/−20 nm filter. Samples were measured individual and a pool on logarithmic scale.

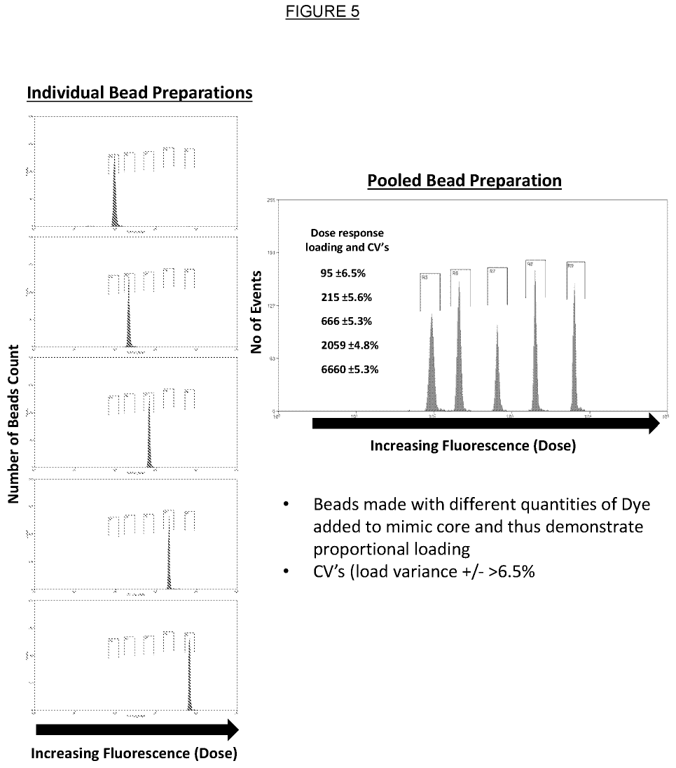

As shown in FIG. 5, clear distinct populations were seen for each of the relative loading samples, showing increases in fluorescence exactly proportional to the relative AMF.HCl bead loading with CV's <6.5%. This means distinct amounts of a chemical compounds can be accurately loaded onto beads allowing quantitative dose responses to be performed in activity assays.

### EXAMPLE 3: ENZYME ACTIVITY MEASUREMENT IN MICRO-DROPLETS

500 μl of —COOH magnetic beads (Bang Beads Inc) were washed 5×1 ml with 10 mM MOPS pH 5.5, 150 mM NaCl and then finally resuspended in 500 μl of the same buffer. Oligos 1 and 2 were each resuspended in Nuclease free water to a concentration of 100 μM.
Oligo 1—/5AmMC6/ATGC/iFluroT/ACGTGCATCCAAGCA/3IABkFQ/
Oligo 2—TGC TTG GAT GCA CGT AGC AT

5AmC6=C6 Amine Linker, iFluroT is a FITC-Fused dT, 3IAbkDQ is a Black Hole Quencher on the 3′ End. Underlined is the BstCl Restriction Enzyme Site.

Oligos 1 and 2 (for bead attachment) were first annealed by mixing equal ratios of each oligo in 100 μl total volume, consisting of 40 μl each oligo, 10 μl of 10×T4 DNA ligase buffer (50 mM Tris-HCl, 10 mM MgCl₂, 10 mM Dithiothreitol, 1 mM ATP, pH 7.5) and 10 μl of Nuclease free water. The mixture was heated to 95° C. in a hot block for 10 minutes and then allowed to cool to room temperature (25° C.) slowly by switching off the block but leaving the sample in the aluminium block.

For double-stranded oligo coupling to the beads: 30 mg of EDC was dissolved in 400 μl of 10 mM MES pH 5.5, 150 mM NaCl. Beads were pulled down with the magnet and then resuspended in the EDC-containing buffer. Once resuspended, the oligo mixture was added and incubation performed with end-over-end mixing at 37° C. for 2 hours.

The beads were washed 5× with 1 ml of 10 mM Tris 1 mM, EDTA pH 7.5 buffer then 2×1 ml with water.

Digest samples with and without inhibitors were set up: Each reaction was in 25 μl, 2.5 μl of Outsmart buffer 3.1 (New England Biolabs, UK), 17.5 μl of beads in water which was then made up to 25 μl with inhibitor and/or water added in order, enzyme added last in every case.
Sample 1+5 μl Nuclease Free Water no enzyme
Sample 2+4 μl Nuclease free water and 1 μl of BstCl
Sample 3+3.5 μl Nuclease free water, 0.5 μl 0.5M EDTA (inhibitor 1) and 1 μl of BstCl
Sample 4+3.5 μl Nuclease free water, 50 mM Spermidine (inhibitor 2, Sigma Aldrich) and 1p1 of BstCl

Samples were immediately emulsified; emulsification was performed using a vortex in bulk with 200p1 of mineral oil (73% Tegosoft DEC, Evonik, 7% Abil EM 90, Evonik and 10% Light mineral oil, Sigma Aldrich). This step can also be performed using a microdroplet-generating device.

The emulsified mixtures containing the reaction/bead-containing microdroplets were incubated with mixing for 30 minutes at 37° C. and then the emulsion broken. 500 μl of 100% ethanol was added and samples were then centrifuged for 1 minute at 14000 g to pellet the beads. These were then washed 3× with 1 ml of 10 mM Tris, 1 mM EDTA pH 7.5 buffer and resuspended in 500 μl of the same buffer.

The beads were then diluted to be roughly 1 million/ml by diluting 20-fold in buffer and run on a Cytoflex flow cytometer (Beckman Coulter). The machine was calibrated to detect small particles (i.e. bacteria). The excitation was at 488 nM with detection at 525 nm+/−40 nm; 10,000 events were detected and the average fluorescence plotted.

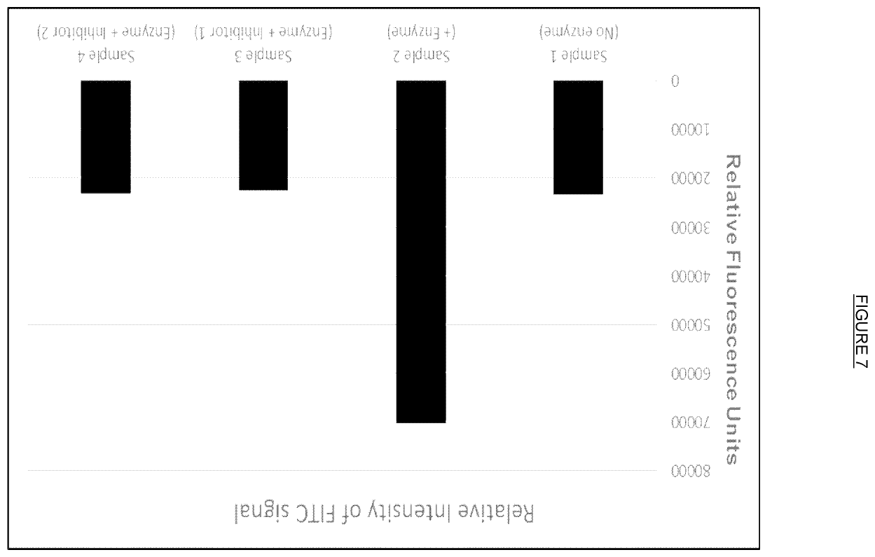

The results are shown in FIG. 7. Sample 2 was active showing 3 times the control signal suggesting cleavage of the oligo retained on the beads and in the microdroplets, while samples with inhibitors and negative control (no enzyme) were similar, showing that inhibition of an enzyme that cleaves a bead-retained target substrate can be performed and clearly detected using the microdroplet incubation system.

It will be appreciated that fluorescent/non-fluorescent beads can be easily be separated by FACS. Thus, initial loading of the beads with compound would permit encoded identification of the specific inhibitor compounds and their respective bead loading.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1-83. (canceled)

84. An encoded chemical library microbead, which microbead has immobilized thereon and/or therein: (i) an encoding tag; and (ii) a target assay system reporter moiety, wherein the reporter moiety exists in a first state in the absence of activity against the target and in a second state in the presence of said activity, and wherein said microbead further comprises a clonal population of one or more chemical structure(s) releasably linked thereto and encoded by said tag.

85. The microbead of claim 84, wherein said encoding tag also encodes the target assay system reporter moiety.

86. The microbead of claim 84, wherein said encoding tag also encodes the target.

87. The microbead of claim 84, wherein the chemical structures are present at a loading of between 1 and 1013 molecules per microbead.

88. The microbead of claim 84, wherein the microbead comprises non-DNA tags, non-RNA tags, modified nucleic acid tags, peptide tags, light-based barcodes (e.g. quantum dots), RFID tags, reporter chemicals linked by click chemistry and mass spectrometry-decodable tags.

89. The microbead of claim 84, wherein the chemical structures are releasably linked to the microbead by a cleavable linker.

90. The microbead of claim 89, wherein the linker is scarless, such that the chemical structure(s) can be cleaved from the microbead in a form in which they are completely or substantially free of linker residues.

91. The microbead of claim 84, wherein the chemical structures are indirectly or directly linked to the microbead.

92. The microbead of claim 84, wherein the target assay system reporter moiety is substrate and chemical structures which function as chromophore coatings can be identified by decoding the tags of microbeads having chromatic reporter moieties.

93. The microbead of claim 84, wherein the target assay system reporter moiety is substrate and chemical structures which function as a substrate coating can be identified by decoding the tags of microbeads having coated reporter moieties.

94. A chemical library microcompartment which contains a microbead as defined in claim 84 and a solvent, for example an aqueous solvent.

95. An encoded chemical library (ECL) comprising a plurality of microcompartments as defined in claim 93, wherein each of the microcompartments contains a different chemical structure.

96. The ECL of claim 95, which comprises a number n of different clonal populations of chemical structures, each clonal population being confined to n discrete library microcompartments.

97. A method for screening an ECL as defined in claim 95 for chemical structures having activity against a target, the method comprising the steps of:
(a) providing said ECL;
(b) releasing the chemical structures from the microbeads to produce a plurality of free, tagless chemical structures (TCSs) dissolved in the solvent and contained within microcompartments together with the microbeads from which they were released, such that a spatial association between each TCS and its encoding tag is maintained;
(c) assaying the TCSs by incubating the ECL microcompartments of step (b) under conditions such that the state of the reporter moieties immobilized in or on the microbeads contained therein is determined by the level of activity against the target;
(d) releasing the assayed microbeads by opening the microcompartments; and
(e) screening the released and assayed microbeads by determining the state of the reporter moieties, whereby chemical structures having activity against the target can be identified by decoding the tags of microbeads having reporter moieties in the second state.

98. The method of claim 97 wherein step (d) further comprises stopping the incubation, for example by heat denaturation, freezing, addition of inhibitors or breaking of the microcompartments.

99. The method of claim 98 wherein the microcompartments are broken by centrifugation, sonication and/or filtration or by the addition of solvents and/or surfactants.

100. The method of claim 97 further comprising the step of isolating the microbeads released in step (d) during or prior to screening step (e).

101. The method of claim 97 wherein the screening step comprises fractionation and/or selection of the released and assayed microbeads.

102. The method of claim 97 wherein the screening step (e) comprises determining the level of activity against the target by measuring the ratio of reporter moieties in the first state to the second state.

103. The method of claim 97 wherein the microbead comprises a clonal population of a plurality of chemical structures and said encoding tag also encodes the loading of the chemical structures, and wherein the screening step (e) comprises determining the level of activity against the target by correlating the loading of the chemical structures with the ratio of reporter moieties in the first state to the second state.


SEQUENCE LISTING (2 sequences)

SEQ ID NO: 1 - DNA, 20 bp, Artificial Sequence
  Test oligonucleotide for enzyme activity measurement in micro-droplets
  misc_feature (1)..(1): C6 amine linker, specifically 5AmC6
  misc_feature (5)..(5): dT fused to FITC, specifically “iFluorT”
  misc_feature (9)..(15): BstCI restriction enzyme site
  misc_feature (20)..(20): Black hole quencher on the 3′ end of the oligo, specifically 3IAbkFQ
atgctacgtgcatccaagca

SEQ ID NO: 2 - DNA, 20 bp, Artificial Sequence
  Test oligonucleotide for enzyme activity measurement in micro-droplets
tgcttggatgcacgtagcat